(19) United States
(12) Patent Application Publication
(10) Pub. No.: US 2022/0395626 A1
(43) Pub. Date: Dec. 15, 2022

(54) SUCTION AND IRRIGATION VALVE FOR A ROBOTIC SURGICAL SYSTEM AND RELATED MATTERS

(71) Applicant: Cilag GmbH International, Zug (CH)

(72) Inventors: Kris E. Kallenberger, Cincinnati, OH (US); Robert P. Kruth, Fort Wayne, IN (US); Niko Murrell, Cincinnati, OH (US); David C. Perdue, Cincinnati, OH (US)

(21) Appl. No.: 17/345,617

(22) Filed: Jun. 11, 2021

(51) Int. Cl. A61M 1/00; A61B 34/00; A61B 34/30

(52) U.S. Cl. CPC A61M 1/743; A61M 1/77; A61B 34/71; A61B 34/30

(57) ABSTRACT

A suction and irrigation valve for a robotic surgical system includes a shaft assembly, a rotary drive, and a valve assembly. The valve assembly is operatively connected to the rotary drive member and includes a valve body and a valve plug. The valve body has a first fluid inlet, a second fluid inlet, and an outlet. The valve plug is received with the valve body and is configured to rotate about a plug axis relative to the valve body to a first position, a second position, and a third position. The valve plug has at least a first conduit configured to selectively fluidly communicate with each of the outlet and at least one of the first or second fluid inlets.

### BACKGROUND

A variety of surgical instruments include an end effector for use in conventional medical treatments and procedures conducted by a medical professional operator, as well as applications in robotically assisted surgeries. Such surgical instruments may be directly gripped and manipulated by a surgeon or incorporated into robotically assisted surgery. In the case of robotically assisted surgery, the surgeon may operate a master controller to remotely control the motion of such surgical instruments at a surgical site. The controller may be separated from the patient by a significant distance (e.g., across the operating room, in a different room, or in a completely different building than the patient). Alternatively, a controller may be positioned quite near the patient in the operating room. Regardless, the controller may include one or more hand input devices (such as joysticks, exoskeletol gloves, master manipulators, or the like), which are coupled by a servo mechanism to the surgical instrument. In one example, a servo motor moves a manipulator supporting the surgical instrument based on the surgeon's manipulation of the hand input devices. During the surgery, the surgeon may employ, via a robotic surgical system, a variety of surgical instruments including an ultrasonic blade, a surgical stapler, a tissue grasper, a needle driver, an electrosurgical cautery probes, etc. Each of these structures performs functions for the surgeon, for example, cutting tissue, coagulating tissue, holding or driving a needle, grasping a blood vessel, dissecting tissue, or cauterizing tissue.

Examples of surgical instruments include suction-irrigation devices. Suction-irrigation devices are configured to apply at least one of suction or irrigation to the surgical site, such as for flushing of fluid and debris at the surgical site via irrigation and for removal of fluid and debris from the surgical site via suction. In this respect the suction-irrigation devices are configured to connect to a vacuum source for suction and a fluid source for irrigation, although such sources may be stored locally within the suction-irrigation device. While irrigation may be directed to the surgical site separately from suction, in some examples irrigation may occur simultaneously with suction. Moreover, during procedures, the medical operator may select the suction or irrigation as desired. Examples of suction-irrigation devices may simply perform suction and irrigation or be incorporated into other surgical instruments for added functionality.

Additional examples of other surgical instruments include surgical staplers. Some such staplers are operable to clamp down on layers of tissue, cut through the clamped layers of tissue, and drive staples through the layers of tissue to substantially seal the severed layers of tissue together near the severed ends of the tissue layers. Examples of surgical staplers and associated features are disclosed in U.S. Pat. No. 7,404,508, entitled “Surgical Stapling and Cutting Device,” issued Jul. 29, 2008; U.S. Pat. No. 7,434,715, entitled “Surgical Stapling Instrument Having Multistroke Firing with Opening Lockout,” issued Oct. 14, 2008; U.S. Pat. No. 7,721,930, entitled “Disposable Cartridge with Adhesive for Use with a Stapling Device,” issued May 25, 2010; U.S. Pat. No. 8,408,439, entitled “Surgical Stapling Instrument with An Articulatable End Effector,” issued Apr. 2, 2013; U.S. Pat. No. 8,453,914, entitled “Motor-Driven Surgical Cutting Instrument with Electric Actuator Directional Control Assembly,” issued Jun. 4, 2013; U.S. Pat. No. 9,186,142, entitled “Surgical Instrument End Effector Articulation Drive with Pinion and Opposing Racks,” issued on Nov. 17, 2015; U.S. Pat. No. 9,795,379, entitled “Surgical Instrument with Multi-Diameter Shaft,” issued Oct. 24, 2017; U.S. Pat. No. 9,808,248, entitled “Installation Features for Surgical Instrument End Effector Cartridge,” issued Nov. 7, 2017; U.S. Pat. No. 10,092,292, entitled “Staple Forming Features for Surgical Stapling Instrument,” issued Oct. 9, 2018; U.S. Pat. No. 9,717,497, entitled “Lockout Feature for Movable Cutting Member of Surgical Instrument,” issued Aug. 1, 2017; U.S. Pat. No. 9,517,065, entitled “Integrated Tissue Positioning and Jaw Alignment Features for Surgical Stapler,” issued Dec. 13, 2016; U.S. Pat. No. 9,622,746, entitled “Distal Tip Features for End Effector of Surgical Instrument,” issued Apr. 18, 2017; and U.S. Pat. No. 8,210,411, entitled “Motor-Driven Surgical Instrument,” issued Jul. 3, 2012. The disclosure of each of the above-cited U.S. Patents is incorporated by reference herein in its entirety.

Still additional examples of such other surgical instruments include an ultrasonic surgical instrument with end effector having a blade element that vibrates at ultrasonic frequencies to cut and/or seal tissue (e.g., by denaturing proteins in tissue cells). These instruments include one or more piezoelectric elements that convert electrical power into ultrasonic vibrations, which are communicated along an acoustic waveguide to the blade element. The precision of cutting and coagulation may be controlled by the operator's technique and adjusting the power level, blade edge angle, tissue traction, and blade pressure. The power level used to drive the blade element may be varied (e.g., in real time) based on sensed parameters such as tissue impedance, tissue temperature, tissue thickness, and/or other factors. Some instruments have a clamp arm and clamp pad for grasping tissue with the blade element. Examples of ultrasonic surgical instruments and related concepts are disclosed in U.S. Pub. No. 2006/0079874, entitled “Tissue Pad for Use with an Ultrasonic Surgical Instrument,” published Apr. 13, 2006, now abandoned; U.S. Pub. No. 2007/0191713, entitled “Ultrasonic Device for Cutting and Coagulating,” published Aug. 16, 2007, now abandoned; U.S. Pub. No. 2008/0200940, entitled “Ultrasonic Device for Cutting and Coagulating,” published Aug. 21, 2008, now abandoned, U.S. Pat. No. 9,949,785, entitled “Ultrasonic Surgical Instrument with Electrosurgical Feature,” issued Apr. 24, 2018,; and U.S. Pat. No. 8,663,220, entitled “Ultrasonic Surgical Instruments,” issued Mar. 4, 2014. The disclosure of each of the above-cited U.S. Patent Publications and U.S. Patents is incorporated by reference herein in its entirety.

While several surgical instruments and systems have been made and used, it is believed that no one prior to the inventors has made or used the invention described in the appended claims.

### BRIEF DESCRIPTION OF THE DRAWINGS

While the specification concludes with claims which particularly point out and distinctly claim this technology, it is believed this technology will be better understood from the following description of certain examples taken in conjunction with the accompanying drawings, in which like reference numerals identify the same elements and in which:

FIG. 1 depicts a perspective view of a first example of a table-based robotic system configured for a laparoscopic procedure;

FIG. 2 depicts a perspective view of a second example of a table-based robotic system;

FIG. 3 depicts an end elevational view of the table-based robotic system of FIG. 2;

FIG. 4 depicts the end elevational view of the table-based robotic system of FIG. 3 including a pair of exemplary robotic arms;

FIG. 5 depicts a partially exploded perspective view of the robotic arm of FIG. 4 having an instrument driver and a first exemplary surgical instrument;

FIG. 6A depicts a side elevational view of the surgical instrument of FIG. 5 in a retracted position;

FIG. 6B depicts the side elevational view the surgical instrument similar to FIG. 6A, but in an extended position;

FIG. 7 depicts a perspective view of a second exemplary surgical instrument having a valve adapter for selectively directing suction and irrigation;

FIG. 8 depicts an enlarged rear perspective view of the valve adapter of FIG. 7;

FIG. 9 depicts an enlarged rear perspective view of the valve adapter of FIG. 7 and an example of a spool valve assembly;

FIG. 10 depicts an enlarged front perspective view of the valve adapter of FIG. 7 having a portion of a housing removed for greater clarity;

FIG. 11 depicts a front perspective view of the valve assembly of FIG. 9;

FIG. 12 depicts a front perspective view of the spool valve assembly of FIG. 9 having a housing removed for greater clarity;

FIG. 13A depicts a cross-sectional view of the spool valve assembly of FIG. 12 taken along section line 13A-13A of FIG. 12 with a vacuum spool valve plug in a closed vacuum position and a fluid spool valve plug in a closed fluid position such than neither a vacuum inlet nor a fluid inlet is in fluid communication with an outlet;

FIG. 13B depicts the cross-sectional view of the spool valve assembly similar to FIG. 13A, but showing the fluid spool valve plug in an open fluid position such that the fluid inlet is in fluid communication with the outlet;

FIG. 13C depicts the cross-sectional view of the spool valve assembly similar to FIG. 13A, but showing the vacuum spool valve plug in an open vacuum position such that the vacuum inlet is in fluid communication with the outlet;

FIG. 14 depicts a front perspective view of an example of a horizontal stopcock valve assembly for selectively directing suction and irrigation with the surgical instrument of FIG. 7;

FIG. 15 depicts a rear perspective view of the horizontal stopcock valve assembly of FIG. 14;

FIG. 16 depicts a partially exploded perspective view of the horizontal stopcock valve assembly of FIG. 14;

FIG. 17 depicts a top view of the horizontal stopcock valve assembly of FIG. 14;

FIG. 18A depicts a cross-sectional view of the horizontal stopcock valve assembly of FIG. 14 taken along section line 18A-18A of FIG. 14 showing an exemplary stopcock valve plug of the horizontal stopcock valve assembly in a first position such that neither a vacuum inlet nor a fluid inlet is in fluid communication with an outlet;

FIG. 18B depicts the cross-section view of the horizontal stopcock valve assembly similar to FIG. 18A, but showing the stopcock valve plug in a second position such that the vacuum inlet is in fluid communication with the fluid inlet;

FIG. 18C depicts the cross-section view of the horizontal stopcock valve assembly similar to FIG. 18B, but showing the stopcock valve plug in a third position such that neither the vacuum inlet nor the fluid inlet is in fluid communication with the outlet;

FIG. 18D depicts the cross-section view of the horizontal stopcock valve assembly similar to FIG. 18C, but showing the stopcock valve plug in a fourth position such that the vacuum inlet is in fluid communication with the outlet;

FIG. 18E depicts the cross-section view of the horizontal stopcock valve assembly similar to FIG. 18D, but showing the stopcock valve plug in a fifth position such that neither the vacuum inlet nor the fluid inlet is in fluid communication with the outlet;

FIG. 18F depicts the cross-section view of the horizontal stopcock valve assembly similar to FIG. 18E, but showing the stopcock valve plug in a sixth position such that the fluid inlet is in fluid communication with the outlet;

FIG. 19 depicts a front perspective view of an example of an axial stopcock valve assembly for selectively directing suction and irrigation with the surgical instrument of FIG. 7;

FIG. 20 depicts a rear perspective view of the axial stopcock valve assembly of FIG. 19;

FIG. 21 depicts an exploded perspective view of the axial stopcock valve assembly of FIG. 19;

FIG. 22A depicts a sectional top view of the axial stopcock valve assembly of FIG. 19 having various features hidden for greater clarity and showing an exemplary stopcock valve plug in a first position such that neither a vacuum inlet nor a fluid inlet is in fluid communication with an outlet;

FIG. 22B depicts the sectional top view of the axial stopcock valve assembly similar to FIG. 22A, but showing the fluid inlet in fluid communication with the outlet;

FIG. 22C depicts the sectional top view of the axial stopcock valve assembly similar to FIG. 22B, but showing neither the vacuum inlet nor the fluid inlet in fluid communication with the outlet;

FIG. 22D depicts the sectional top view of the axial stopcock valve assembly similar to FIG. 22C, but showing the vacuum inlet in fluid communication with the outlet;

FIG. 22E depicts the sectional top view of the axial stopcock valve assembly similar to FIG. 22D, but showing neither the vacuum inlet nor the fluid inlet in fluid communication with the outlet;

FIG. 22F depicts the sectional top view of the axial stopcock valve assembly similar to FIG. 22E, but showing the vacuum inlet in fluid communication with the fluid inlet;

FIG. 23 depicts a front perspective view of an example of a vertical stopcock valve assembly for selectively directing suction and irrigation with the surgical instrument of FIG. 7;

FIG. 24 depicts an exploded perspective view of the vertical stopcock valve assembly of FIG. 23;

FIG. 25 depicts a top view of the vertical stopcock valve assembly of FIG. 23 with an exemplary stopcock valve plug in a first position;

FIG. 25A depicts a cross-sectional view of the vertical stopcock valve assembly of FIG. 25 taken along section line 25A-25A of FIG. 25 showing the stopcock valve plug in the first position such that nether a vacuum inlet nor a fluid inlet is in fluid communication with an outlet;

FIG. 26 depicts a top view of the vertical stopcock valve assembly of FIG. 23 with the stopcock valve plug in a second position;

FIG. 26A depicts a cross-sectional view of the vertical stopcock valve assembly of FIG. 26 taken along section line 26A-26A of FIG. 26 showing the stopcock valve plug in the first position such that the vacuum inlet is in fluid communication with the fluid inlet;

FIG. 27 depicts a top view of the vertical stopcock valve assembly of FIG. 23 with the stopcock valve plug in a third position;

FIG. 27A depicts a cross-sectional view of the vertical stopcock valve assembly of FIG. 27 taken along section line 27A-27A of FIG. 27 showing the stopcock valve plug in the first position such that nether the vacuum inlet nor the fluid inlet is in fluid communication with the outlet;

FIG. 28 depicts a top view of the vertical stopcock valve assembly of FIG. 23 with the stopcock valve plug in a fourth position;

FIG. 28A depicts a cross-sectional view of the vertical stopcock valve assembly of FIG. 28 taken along section line 28A-28A of FIG. 28 showing the stopcock valve plug in the fourth position such that the vacuum inlet is in fluid communication with the outlet;

FIG. 29 depicts a top view of the vertical stopcock valve assembly of FIG. 23 with the stopcock valve plug in a fifth position;

FIG. 29A depicts a cross-sectional view of the vertical stopcock valve assembly of FIG. 29 taken along section line 29A-29A of FIG. 29 showing the stopcock valve plug in the fifth position such that nether the vacuum inlet nor the fluid inlet is in fluid communication with the outlet;

FIG. 30 depicts a top view of the vertical stopcock valve assembly of FIG. 23 with the stopcock valve plug in a sixth position;

FIG. 30A depicts a cross-sectional view of the vertical stopcock valve assembly of FIG. 30 taken along section line 30A-30A of FIG. 30 showing the stopcock valve plug in the sixth position such that the fluid inlet is in fluid communication with the outlet;

FIG. 31 depicts an enlarged rear perspective view of a third exemplary surgical instrument having a first example of a reusable instrument housing and a first example of a disposable valve assembly;

FIG. 32 depicts an enlarged partially exploded rear perspective view of the surgical instrument of FIG. 31 with the disposable valve assembly removed from the reusable instrument housing;

FIG. 33 depicts an enlarged top perspective view of the disposable valve assembly of FIG. 31 removed from the reusable instrument housing of FIG. 31 with various features removed for greater clarity;

FIG. 34 depicts an enlarged top perspective view of the disposable valve assembly of FIG. 31 connected to the reusable instrument housing of FIG. 31 with various features removed for greater clarity;

FIG. 35 depicts a schematic sectional top view of the disposable valve assembly of FIG. 31 including an example of a trumpet valve assembly connected to the reusable instrument housing of FIG. 31;

FIG. 36 depicts a schematic sectional top view of the disposable valve assembly of FIG. 31 including an example of a pinch valve assembly connected to the reusable instrument housing of FIG. 31;

FIG. 37 depicts an enlarged rear perspective view of a fourth exemplary surgical instrument having a second example of a reusable instrument housing and a second example of a disposable valve assembly; and

FIG. 38 depicts an enlarged partially exploded rear perspective view of the surgical instrument of FIG. 37 with the disposable valve assembly removed from the reusable instrument housing.

The drawings are not intended to be limiting in any way, and it is contemplated that various embodiments of the technology may be carried out in a variety of other ways, including those not necessarily depicted in the drawings. The accompanying drawings incorporated in and forming a part of the specification illustrate several aspects of the present technology, and together with the description serve to explain the principles of the technology; it being understood, however, that this technology is not limited to the precise arrangements shown.

### DETAILED DESCRIPTION

The following description of certain examples of the technology should not be used to limit its scope. Other examples, features, aspects, embodiments, and advantages of the technology will become apparent to those skilled in the art from the following description, which is by way of illustration, one of the best modes contemplated for carrying out the technology. As will be realized, the technology described herein is capable of other different and obvious aspects, all without departing from the technology. Accordingly, the drawings and descriptions should be regarded as illustrative in nature and not restrictive.

It is further understood that any one or more of the teachings, expressions, embodiments, examples, etc. described herein may be combined with any one or more of the other teachings, expressions, embodiments, examples, etc. that are described herein. The following-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

For clarity of disclosure, the terms “proximal” and “distal” are defined herein relative to a human or robotic operator of the surgical instrument. The term “proximal” refers the position of an element closer to the human or robotic operator of the surgical instrument and further away from the surgical end effector of the surgical instrument. The term “distal” refers to the position of an element closer to the surgical end effector of the surgical instrument and further away from the human or robotic operator of the surgical instrument. It will be further appreciated that, for convenience and clarity, spatial terms such as “clockwise,” “counterclockwise,” “longitudinal,” “inner,” “outer,” and “upper,” also are used herein for reference to relative positions and directions. Such terms are used below with reference to views as illustrated for clarity and are not intended to limit the invention described herein.

Aspects of the present examples described herein may be integrated into a robotically-enabled medical system, including as a robotic surgical system, capable of performing a variety of medical procedures, including both minimally invasive, such as laparoscopy, and non-invasive, such as endoscopy, procedures. Among endoscopy procedures, the robotically-enabled medical system may be capable of performing bronchoscopy, ureteroscopy, gastroscopy, etc.

In addition to performing the breadth of procedures, the robotically-enabled medical system may provide additional benefits, such as enhanced imaging and guidance to assist the medical professional. Additionally, the robotically-enabled medical system may provide the medical professional with the ability to perform the procedure from an ergonomic position without the need for awkward arm motions and positions. Still further, the robotically-enabled medical system may provide the medical professional with the ability to perform the procedure with improved ease of use such that one or more of the instruments of the robotically-enabled medical system may be controlled by a single operator.

### I. Exemplary Robotically-Enabled Medical System

FIG. 1 shows an exemplary robotically-enabled medical system, including a first example of a table-based robotic system (10). Table-based robotic system (10) of the present example includes a table system (12) operatively connected to a surgical instrument (14) for a diagnostic and/or therapeutic procedure in the course of treating a patient. Such procedures may include, but are not limited, to bronchoscopy, ureteroscopy, a vascular procedure, and a laparoscopic procedure. To this end, surgical instrument (14) is configured for a laparoscopic procedure, although it will be appreciated that any instrument for treating a patient may be similarly used. At least part of table-based robotic system (10) may be constructed and operable in accordance with at least some of the teachings of any of the various patents, patent application publications, and patent applications that are cited herein.

### A. First Exemplary Table-Based Robotic System

With respect to FIG. 1, table-based robotic system (10) includes table system (12) having a platform, such as a table (16), with a plurality of carriages (18) which may also be referred to herein as “arm supports,” respectively supporting the deployment of a plurality of robotic arms (20). Table-based robotic system (10) further includes a support structure, such as a column (22), for supporting table (16) over the floor. Table (16) may also be configured to tilt to a desired angle during use, such as during laparoscopic procedures. Each robotic arm (20) includes an instrument driver (24) configured to removably connect to and manipulate surgical instrument (14) for use. In alternative examples, instrument drivers (24) may be collectively positioned in a linear arrangement to support the instrument extending therebetween along a “virtual rail” that may be repositioned in space by manipulating the one or more robotic arms (20) into one or more angles and/or positions. In practice, a C-arm (not shown) may be positioned over the patient for providing fluoroscopic imaging.

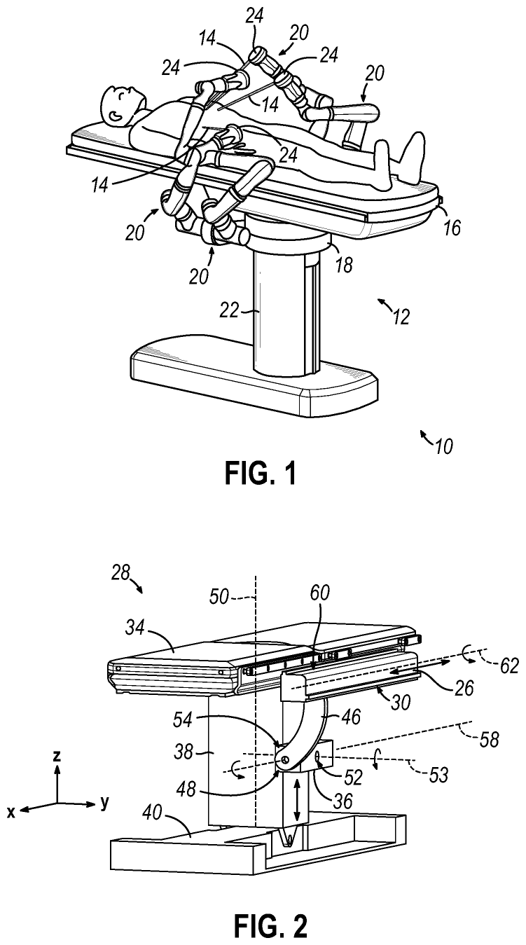

In the present example, column (22) includes carriages (18) arranged in a ring-shaped form to respectively support one or more robotic arms (20) for use. Carriages (18) may translate along column (22) and/or rotate about column (22) as driven by a mechanical motor (not shown) positioned within column (22) in order to provide robotic arms (20) with access to multiples sides of table (16), such as, for example, both sides of the patient. Rotation and translation of carriages (18) allows for alignment of instruments, such as surgical instrument (14) into different access points on the patient. In alternative examples, such as those discussed below in greater detail, table-based robotic system (10) may include a patient table or bed with adjustable arm supports including a bar (26) (see FIG. 2) extending alongside. One or more robotic arms (20) (e.g., via a shoulder with an elbow joint) may be attached to carriages (18), which are vertically adjustable so as to be stowed compactly beneath the patient table or bed, and subsequently raised during use.

Table-based robotic system (10) may also include a tower (not shown) that divides the functionality of table-based robotic system (10) between table (16) and the tower to reduce the form factor and bulk of table (16). To this end, the tower may provide a variety of support functionalities to table (16), such as processing, computing, and control capabilities, power, fluidics, and/or optical and sensor processing. The tower may also be movable so as to be positioned away from the patient to improve medical professional access and de-clutter the operating room. The tower may also include a master controller or console that provides both a user interface for operator input, such as keyboard and/or pendant, as well as a display screen, including a touchscreen, for pre-operative and intra-operative information, including, but not limited to, real-time imaging, navigation, and tracking information. In one example, the tower may include gas tanks to be used for insufflation.

### B. Second Exemplary Table-Based Robotic System

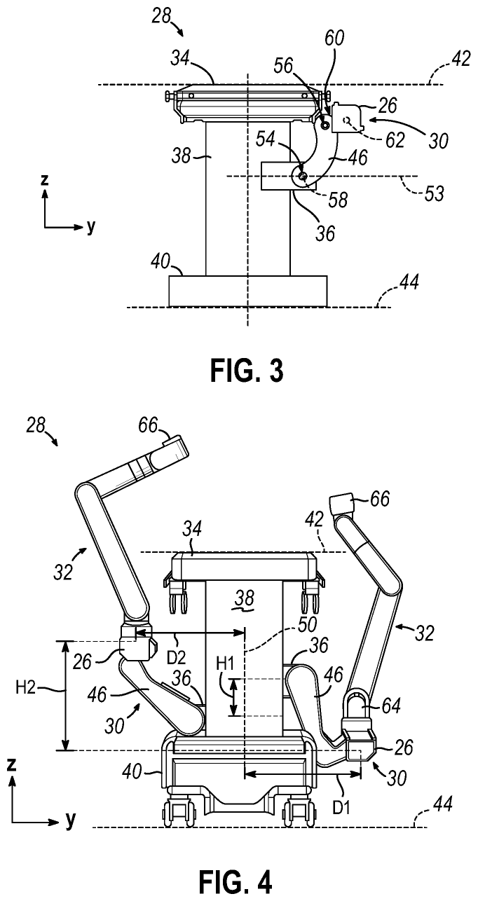

As discussed briefly above, a second exemplary table-based robotic system (28) includes one or more adjustable arm supports (30) including bars (26) configured to support one or more robotic arms (32) relative to a table (34) as shown in FIGS. 2-4. In the present example, a single and a pair of adjustable arm supports (30) are shown, though additional arm supports (30) may be provided about table (34). Adjustable arm support (30) is configured to selectively move relative to table (34) so as to alter the position of adjustable arm support (30) and/or any robotic arms (32) mounted thereto relative to table (34) as desired. Such adjustable arm supports (30) provide high versatility to table-based robotic system (28), including the ability to easily stow one or more adjustable arm supports (30) with robotic arms (32) beneath table (34).

Each adjustable arm support (30) provides several degrees of freedom, including lift, lateral translation, tilt, etc. In the present example shown in FIGS. 2-4, arm support (30) is configured with four degrees of freedom, which are illustrated with arrows. A first degree of freedom allows adjustable arm support (30) to move in the z-direction (“Z-lift”). For example, adjustable arm support (30) includes a vertical carriage (36) configured to move up or down along or relative to a column (38) and a base (40) supporting table (34). A second degree of freedom allows adjustable arm support (30) to tilt about an axis extending in the y-direction. For example, adjustable arm support (30) includes a rotary joint, which allows adjustable arm support (30) to align the bed in a Trendelenburg position. A third degree of freedom allows adjustable arm support (30) to “pivot up” about an axis extending in the x-direction, which may be useful to adjust a distance between a side of table (34) and adjustable arm support (30). A fourth degree of freedom allows translation of adjustable arm support (30) along a longitudinal length of table (34), which extends along the x-direction. Base (40) and column (38) support table (34) relative to a support surface, which is shown along a support axis (42) above a floor axis (44) and in the present example. While the present example shows adjustable arm support (30) mounted to column (38), arm support (30) may alternatively be mounted to table (34) or base (40).

As shown in the present example, adjustable arm support (30) includes vertical carriage (36), a bar connector (46), and bar (26). To this end, vertical carriage (36) attaches to column (38) by a first joint (48), which allows vertical carriage (36) to move relative to column (38) (e.g., such as up and down a first, vertical axis (50) extending in the z-direction). First joint (48) provides the first degree of freedom (“Z-lift”) to adjustable arm support (30). Adjustable arm support (30) further includes a second joint (52), which provides the second degree of freedom (tilt) for adjustable arm support (30) to pivot about a second axis (53) extending in the y-direction. Adjustable arm support (30) also includes a third joint (54), which provides the third degree of freedom (“pivot up”) for adjustable arm support (30) about a third axis (58) extending in the x-direction. Furthermore, an additional joint (56) mechanically constrains third joint (54) to maintain a desired orientation of bar (26) as bar connector (46) rotates about third axis (58). Adjustable arm support (30) includes a fourth joint (60) to provide a fourth degree of freedom (translation) for adjustable arm support (30) along a fourth axis (62) extending in the x-direction.

With respect to FIG. 4, table-based robotic system (28) is shown with two adjustable arm supports (30) mounted on opposite sides of table (34). A first robotic arm (32) is attached to one such bar (26) of first adjustable arm support (30). First robotic arm (32) includes a base (64) attached to bar (26). Similarly, second robotic arm (32) includes base (64) attached to other bar (26). Distal ends of first and second robotic arms (32) respectively include instrument drivers (66), which are configured to attach to one or more instruments such as those discussed below in greater detail.

In one example, one or more robotic arms (32) has seven or more degrees of freedom. In another example, one or more robotic arms (32) has eight degrees of freedom, including an insertion axis (1-degree of freedom including insertion), a wrist (3-degrees of freedom including wrist pitch, yaw and roll), an elbow (1-degree of freedom including elbow pitch), a shoulder (2-degrees of freedom including shoulder pitch and yaw), and base (64) (1-degree of freedom including translation). In one example, the insertion degree of freedom is provided by robotic arm (32), while in another example, such as surgical instrument (14) (see FIG. 6A), the instrument includes an instrument-based insertion architecture.

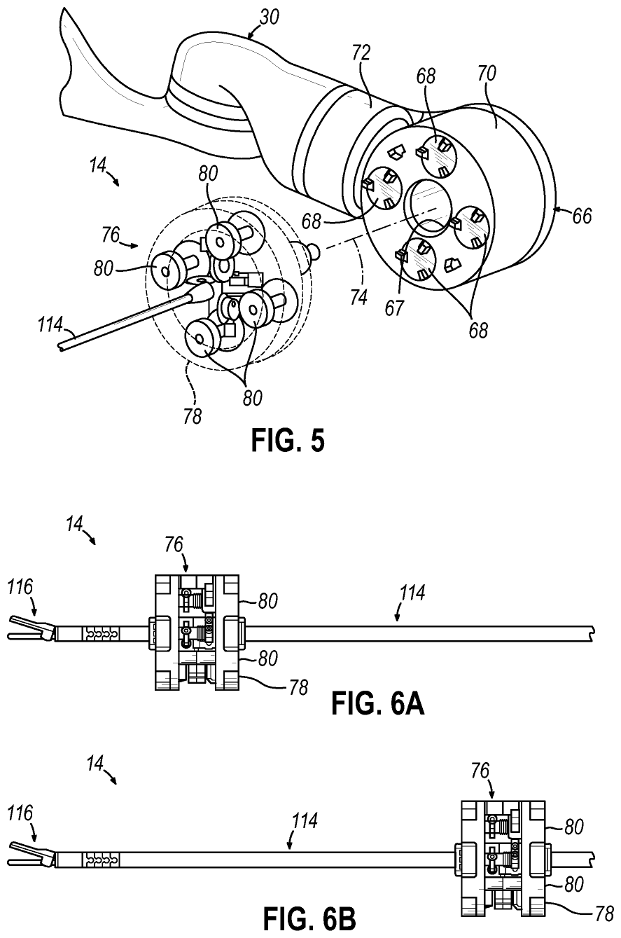

FIG. 5 shows one example of instrument driver (66) in greater detail with surgical instrument (14) removed therefrom. Given the present instrument-based insertion architecture shown with reference to surgical instrument (14), instrument driver (66) further includes a clearance bore (67) extending entirely therethrough so as to movably receive a portion of surgical instrument (14) as discussed below in greater detail. Instrument driver (66) may also be referred to herein as an “instrument drive mechanism,” an “instrument device manipulator,” or an “advanced device manipulator” (ADM). Instruments may be designed to be detached, removed, and interchanged from instrument driver (66) for individual sterilization or disposal by the medical professional or associated staff In some scenarios, instrument drivers (66) may be draped for protection and thus may not need to be changed or sterilized.

Each instrument driver (66) operates independently of other instrument drivers (66) and includes a plurality of rotary drive outputs (68), such as four drive outputs (68), also independently driven relative to each other for directing operation of surgical instrument (14). Instrument driver (66) and surgical instrument (14) of the present example are aligned such that the axes of each drive output (68) are parallel to the axis of surgical instrument (14). In use, control circuitry (not shown) receives a control signal, transmits motor signals to desired motors (not shown), compares resulting motor speed as measured by respective encoders (not shown) with desired speeds, and modulates motor signals to generate desired torque at one or more drive outputs (68).

In the present example, instrument driver (66) is circular with respective drive outputs (68) housed in a rotational assembly (70). In response to torque, rotational assembly (70) rotates along a circular bearing (not shown) that connects rotational assembly (70) to a non-rotational portion (72) of instrument driver (66). Power and controls signals may be communicated from non-rotational portion (72) of instrument driver (66) to rotational assembly (70) through electrical contacts therebetween, such as a brushed slip ring connection (not shown). In one example, rotational assembly (70) may be responsive to a separate drive output (not shown) integrated into non-rotatable portion (72), and thus not in parallel to the other drive outputs (68). In any case, rotational assembly (70) allows instrument driver (66) to rotate rotational assembly (70) and drive outputs (68) in conjunction with surgical instrument (14) as a single unit around an instrument driver axis (74).

Any systems described herein, including table-based robotic system (28), may further include an input controller (not shown) for manipulating one or more instruments. In some embodiments, the input controller (not shown) may be coupled (e.g., communicatively, electronically, electrically, wirelessly and/or mechanically) with an instrument such that manipulation of the input controller (not shown) causes a corresponding manipulation of the instrument e.g., via master slave control. In one example, one or more load cells (not shown) may be positioned in the input controller such that portions of the input controller (not shown) are capable of operating under admittance control, thereby advantageously reducing the perceived inertia of the controller while in use.

In addition, any systems described herein, including table-based robotic system (28) may provide for non-radiation-based navigational and localization means to reduce exposure to radiation and reduce the amount of equipment within the operating room. As used herein, the term “localization” may refer to determining and/or monitoring the position of objects in a reference coordinate system. Technologies such as pre-operative mapping, computer vision, real-time electromagnetic sensor (EM) tracking, and robot command data may be used individually or in combination to achieve a radiation-free operating environment. In other cases, where radiation-based imaging modalities are still used, the pre-operative mapping, computer vision, real-time EM tracking, and robot command data may be used individually or in combination to improve upon the information obtained solely through radiation-based imaging modalities.

### C. Exemplary Surgical Instrument

With respect to FIGS. 5-6B and in cooperation with instrument driver (66) discussed above, surgical instrument (14) includes an elongated shaft assembly (114) and an instrument base (76) with an attachment interface (78) having a plurality of drive inputs (80) configured to respectively couple with corresponding drive outputs (68). Shaft assembly (114) of ultrasonic surgical instrument (14) extends from a center of instrument base (76) with an axis substantially parallel to the axes of the drive inputs (80) as discussed briefly above. With shaft assembly (114) positioned at the center of instrument base (76), shaft assembly (114) is coaxial with instrument driver axis (74) when attached and movably received in clearance bore (67). Thus, rotation of rotational assembly (70) causes shaft assembly (114) of surgical instrument (14) to rotate about its own longitudinal axis while clearance bore (67) provides space for translation of shaft assembly (114) during use.

To this end, FIGS. 5-6B show surgical instrument (14) having the instrument-based insertion architecture as discussed briefly above. Surgical instrument (14) includes elongated shaft assembly (114), end effector (116) connected to and extending distally from shaft assembly (114), and instrument base (76) coupled to shaft assembly (114). Notably, insertion of shaft assembly (114) is grounded at instrument base (76) such that end effector (116) is configured to selectively move longitudinally from a retracted position to an extended position, vice versa, and any desired longitudinal position therebetween. As used herein, the retracted position is shown in FIG. 6A and places end effector (116) relatively close and proximally toward instrument base (76), whereas the extended position is shown in FIG. 6B and places end effector (116) relatively far and distally away from instrument base (76). Insertion into and withdrawal of end effector (116) relative to the patient may thus be facilitated by ultrasonic surgical instrument (14), although it will be appreciated that such insertion into and withdrawal may also occur via adjustable arm supports (30) in one or more examples.

While the present example of instrument driver (66) shows drive outputs (68) arranged in rotational assembly (70) so as to face in a distal direction like distally projecting end effector (116) from shaft assembly (114), an alternative instrument driver (not shown) may include drive output (68) arranged on an alternative rotational assembly (70) to face in a proximal direction, opposite of the distally projecting end effector (116). In such an example, surgical instrument (14) may thus have drive inputs (80) facing distally to attach to instrument drivers (66) facing proximally in an opposite direction from that shown in FIG. 5. The invention is thus not intended to be unnecessarily limited to the particular arrangement of drive outputs (68) and drive inputs (80) shown in the present example and any such arrangement for operatively coupling between drive outputs and inputs (68, 80) may be similarly used.

While various features configured to facilitate movement between end effector (116) and drive inputs (80) are described herein, such features may additionally or alternatively include pulleys, cables, carriages, carriers, such as a kinetic articulating rotating tool (KART), and/or other structures configured to communicate movement along shaft assembly (114). Moreover, while instrument base (76) is configured to operatively connect to instrument driver (66) for driving various features of shaft assembly (114) and/or end effector (116) as discussed below in greater detail, it will be appreciated that alternative examples may operatively connect shaft assembly (114) and/or end effector (116) to an alternative handle assembly (not shown). Such handle assembly (not shown) may include a pistol grip (not shown) in one example, configured to be directly gripped and manipulated by the medical professional for driving various features of shaft assembly (114) and/or end effector (116). The invention is thus not intended to be unnecessarily limited to use with instrument driver (66).

### II. Exemplary Suction-Irrigation Surgical Instrument

In some instances, it may be desirable to use various alternative surgical instruments with robotic systems (10, 28) described above in addition to, or in lieu of, surgical instrument (14). Such alternative surgical instruments may be desirable to provide improved operability and/or functionality when used with robotic systems (10, 28). For instance, as described above, surgical instrument (14) may move between a retracted position and extended position. Additionally, it may be beneficial to translate a portion of surgical instrument (14) along a support structure to provide increased surgical access without increasing the dimensions of surgical instrument (14). As also described above, use of rotational assembly (70) of robotic arm (20, 32) may enable rotation of the entire surgical instrument (14), rather than specific structures of surgical instrument (14) being rotatable.

One such example of these alternative surgical instruments includes a second exemplary surgical instrument (210), which may also be referred to as suction-irrigation surgical instrument (210) and is discussed below in greater detail. Additional examples of alternative surgical instruments and/or associated features for incorporation with robotic systems (10, 28) are described in U.S. patent application Ser. No. 16/946,363, entitled “Articulation Mechanisms for Robotic Surgical Tools,” filed on Jun. 18, 2020; U.S. patent application Ser. No. 17/077,067, entitled “Surgical Instrument and Carrier KART Supporting Ultrasonic Transducer,” filed on Oct. 22, 2020; U.S. patent application Ser. No. 17/077,086, entitled “Carrier KART and Jaw Closure of an Ultrasonic Surgical Instrument,” filed on Oct. 22, 2020; U.S. patent application Ser. No. 17/077,130, entitled “Surgical Instrument with Clamping Sensor Feedback and Related Methods,” filed on Oct. 22, 2020; U.S. patent application Ser. No. 17/077,136, entitled “Surgical Instrument with Non-clamping Sensor Feedback and Related Methods,” filed on Oct. 22, 2020; U.S. patent application Ser. No. 17/077,250, entitled “Ultrasonic Surgical Instrument with a Carrier KART and Reusable Stage,” filed on Oct. 22, 2020; U.S. patent application Ser. No. 17/077,373, entitled “Surgical Instrument with a Carrier KART and Various Communication Cable Arrangements,” filed on Oct. 22, 2020; U.S. patent application Ser. No. 17/077,139, entitled “Ultrasonic Surgical Instrument with a Fixed Transducer Grounding,” filed on Oct. 22, 2020; U.S. patent application Ser. No. 17/077,146, entitled “Ultrasonic Surgical Instrument with a Shaft Assembly and Elongated Waveguide Support Arrangement,” filed on Oct. 22, 2020; U.S. patent application Ser. No. 17/077,152, entitled “Damping Rings for an Ultrasonic Surgical Instrument,” filed on Oct. 22, 2020; U.S. patent application Ser. No. 17/077,110, entitled “Ultrasonic Surgical Instrument with a Mid-shaft Closure System and Related Methods,” filed on Oct. 22, 2020; U.S. patent application Ser. No. 17/076,956, entitled “Surgical Instrument with an Articulatable Shaft Assembly and Dual End Effector Roll,” filed on Oct. 22, 2020; U.S. patent application Ser. No. 17/076,959, entitled “Ultrasonic Surgical Instrument with a Distally Grounded Acoustic Waveguide,” filed on Oct. 22, 2020; and/or U.S. patent application Ser. No. 17/077,098, entitled “Ultrasonic Surgical Instrument with a Multiplanar Articulation Joint,” filed on Oct. 22, 2020. The disclosure of each of the above-cited U.S. Patent Applications is incorporated by reference herein in its entirety. Various features of these alternative examples of surgical instruments may be readily incorporated into a surgical robotic system, such as robotic systems (10, 28), such that the invention is not intended to be unnecessarily limited to these particular alternative surgical instruments discussed herein.

### A. Overview

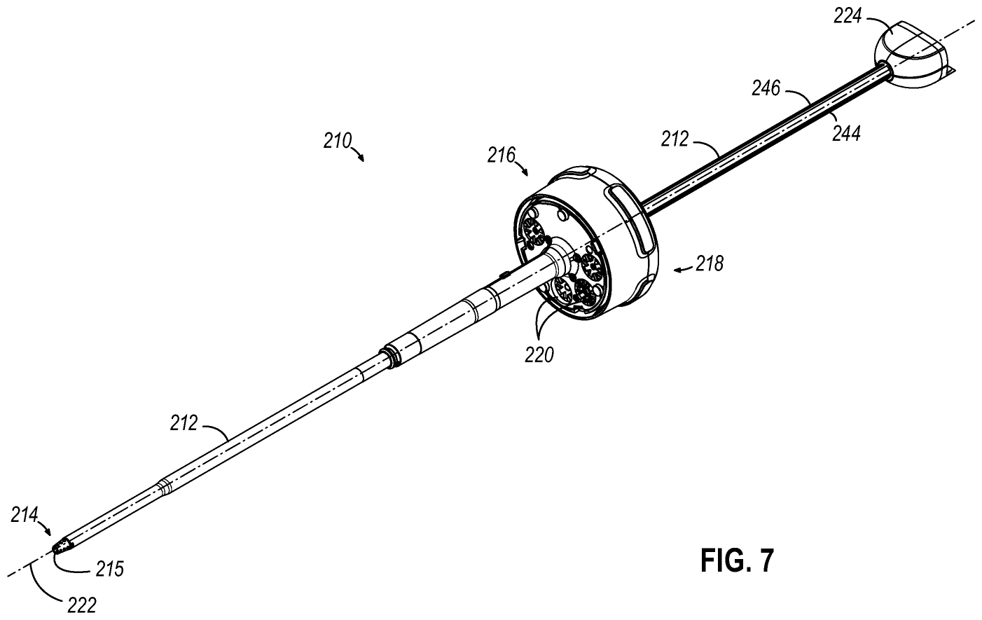

FIG. 7 is an exemplary surgical instrument (210) that may incorporate some or all of the principles of the present disclosure. Surgical instrument (210) may be similar in some respects to any of the instruments described above with reference to FIGS. 1-6B and, therefore, may be used in conjunction with a robotic surgical system, such as robotic systems (10, 28) of FIGS. 1-6B. As illustrated, surgical instrument (210) includes an elongated shaft assembly (212) and an end effector (214) arranged at a distal end of shaft assembly (212).

Surgical instrument (210) can have any of a variety of configurations capable of performing one or more surgical functions. In the present example, surgical instrument (210) is more particularly a suction-irrigation surgical instrument (210) with end effector (214) comprising a distal opening (215) configured to apply suction and/or irrigation to a surgical site. Additionally or alternatively, end effector (214) may comprise other types of instruments requiring opposing jaws such as, but not limited to, surgical staplers (e.g., circular and linear staplers), tissue graspers, surgical scissors, advanced energy vessel sealers, clip appliers, needle drivers, a babcock including a pair of opposed grasping jaws, bipolar jaws (e.g., bipolar Maryland grasper, forceps, a fenestrated grasper, etc.), an endoscope (e.g., a camera), an ultrasonic instrument, an RF instrument, or any combination thereof. etc.

Surgical instrument (210) includes an instrument base (216) with an attachment interface (218) operable similar to instrument base (76) and attachment interface (78) described above. Attachment interface (78) further includes one or more drive inputs (220) for coupling with one or more drive outputs, such as drive outputs (68). Shaft assembly (212) extends from a center of instrument base (216) with an axis substantially parallel to the axes of drive inputs (220) similar to drive inputs (68) as discussed above. Shaft assembly (212) of surgical instrument (14) is thereby configured to rotate about its own longitudinal axis (222) while also longitudinally translating along its axis (222) relative to rotational assembly (70) during use. As will be described in greater detail below, surgical instrument (210) further includes a valve adapter (224). Valve adapter (224) is configured to fluidly couple one or more additional features with end effector (214) via shaft assembly (212), and further to provide operative control of valve adapter (224) by instrument driver (66).

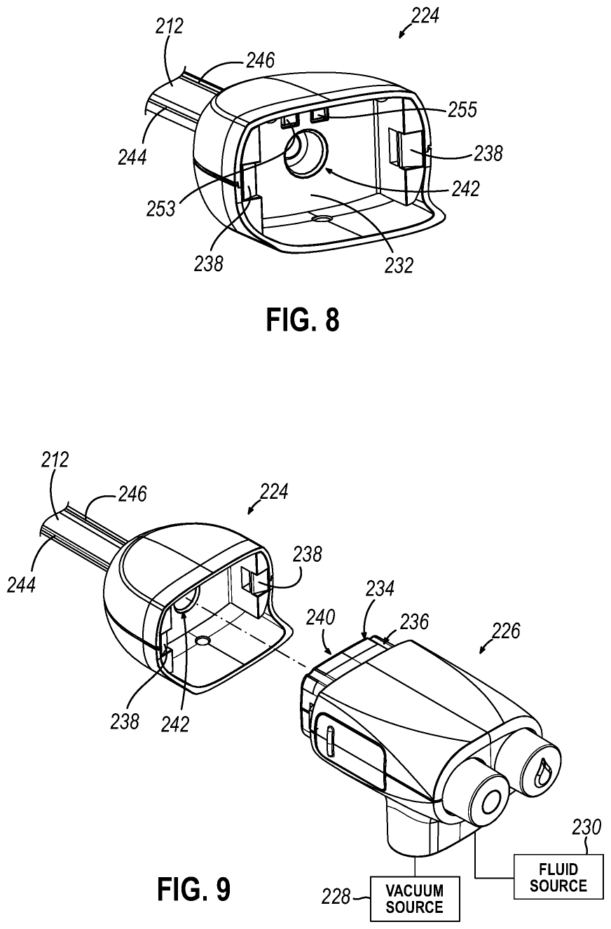

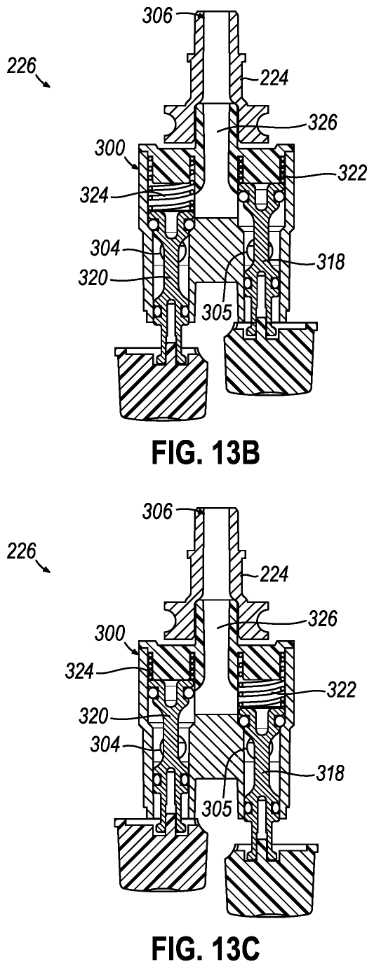

More specifically, depicted in FIGS. 8-9, valve adapter (224) is configured to fluidly and operably couple with a valve assembly (226) or other similarly operable device to provide one or more fluid connections between valve adapter (224), valve assembly (226), and end effector (214). Accordingly, a proximal face (232) of valve adapter (224) is configured to couple with a distal face (234) of valve assembly (226), using latching connectors (236) and latches (238), such that an output (240) of valve assembly (226) fluidly couples with lumen (258) of valve adapter (224) via an input opening (242). The one or more fluid connections may be, for example, a first connection to receive suction from a vacuum (or “suction”) source (228) and a second connection to receive fluid from a fluid (or “irrigation”) source (230). As will be described in greater detail below, each of vacuum source (228) and fluid source (230) may be coupled with a surface of valve assembly (226), and valve assembly (226) may therefore be operable to selectively couple one or both of vacuum source (228) and fluid source (230) to end effector (214) as directed by instrument driver (66). As such, as fluid or suction from vacuum source (228) and fluid source (230) are activated, any such fluid or suction transmits through one or more internal lumens of valve assembly (226), into input opening (242) of valve adapter (224), and flows through inner lumen (258) of valve adapter (224) and into a proximal portion of shaft assembly (212) (see, for example, FIGS. 13A-13C).

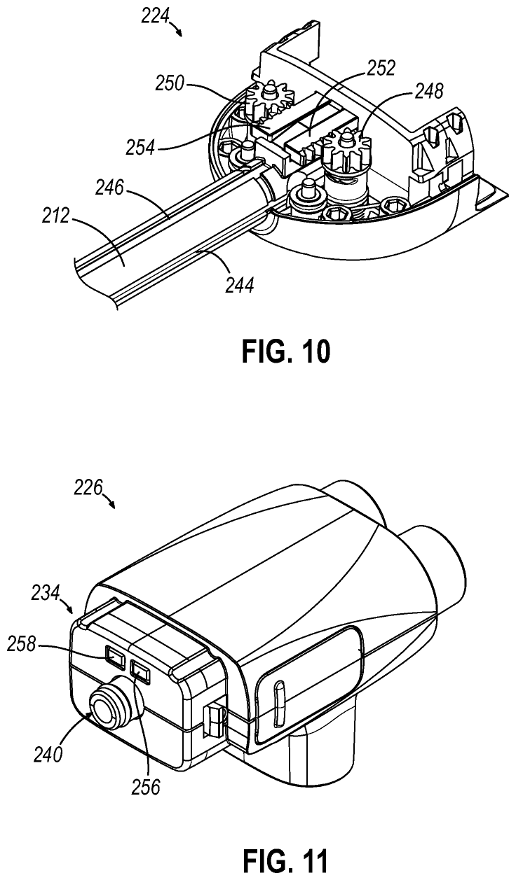

To direct the operation of valve assembly (226), one or more pull cables (244, 246) may be operatively coupled between instrument base (216) and valve adapter (224). Depicted in FIG. 10 is valve adapter (224) having a portion of the exterior housing removed for greater clarity. As shown, pull cables (244, 246) may be selectively operable to direct movements of respective pinions (248, 250), which are operably coupled with respective racks (252, 254). As such, translation of pull cables (244, 246) longitudinally along axis (222) converts the longitudinal movements of pull cables (244, 246) to rotational movement of pinions (248, 250), which is again converted back to longitudinal movement of racks (252, 254). As will be described in greater detail below, racks (252, 254) are further configured to provide input operation to valve assembly (226) by extending and retracting longitudinally through openings (253, 255) of valve adapter (see FIG. 8) and into openings (256, 258) of valve assembly (226) (see FIG. 11) of valve assembly (226).

### B. Spool Valve Assembly

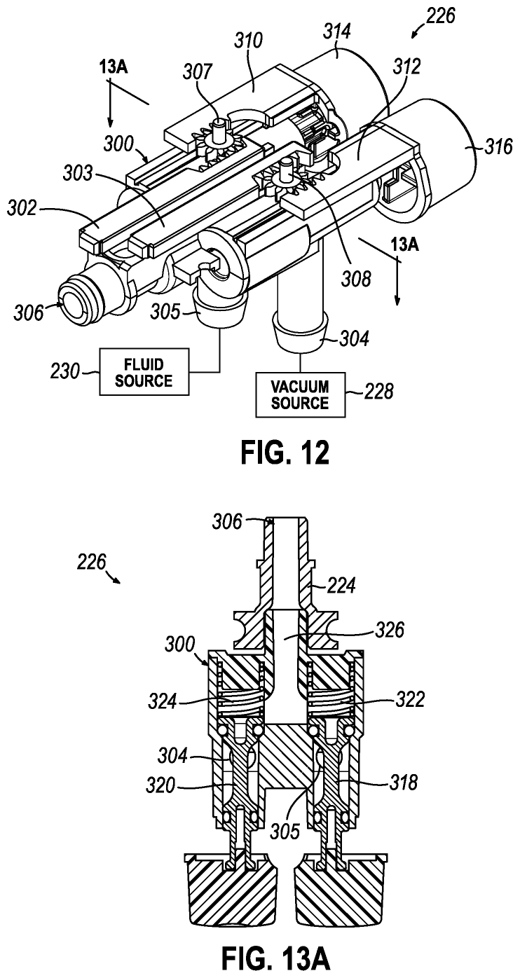

In some versions of surgical instrument (210), valve assembly (226) may be configured and operable as a spool valve assembly (226). Depicted in FIG. 12 is spool valve assembly (226) having a portion of the exterior housing body removed for greater clarity. As shown, spool valve assembly (226) includes a valve body (300) and a pair of racks (302, 303) configured to translate longitudinally as directed by racks (252, 254) of valve adapter (224). Valve body (300) has vacuum and fluid inlet openings (304, 305) as well as an outlet opening (306). Proximal ends of racks (252, 254) of valve adapter (224) are configured to abut against distal ends of racks (302, 303) of spool valve assembly (226) to translate racks (302, 303) longitudinally, which further acts to rotate pinions (307, 308) of spool valve assembly (226). Each pinion (307, 308) is coupled with one of the valve inputs, specifically, the input from vacuum source (228) or the input from fluid source (230). Each pinion (307, 308) is further coupled with a second set of racks (310, 312) which attached to respective fluid and vacuum projections (314, 316). Fluid and vacuum projections (314, 316) are operatively coupled with pinions (307, 308) and configured to translate longitudinally in approximately equal and opposite directions as racks (302, 303) when driven robotically via racks (252, 254). In other words, fluid and vacuum projections (314, 316) generally follow movement of racks (310, 312) and, in this respect, provide a visual indication of valving set for suction or irrigation. In addition or alternatively, robotic operation may be disabled and fluid and vacuum projections (314, 316) may be manually gripped and moved as desired to achieve suction or irrigation.

Accordingly, for example, as first rack (302) translates proximally to rotate first pinion (307), a fluid inlet spool valve plug (318) (see FIGS. 13A-C) from fluid source (230) is opened to thereby couple fluid source (230) with shaft assembly (212). Fluid projection (314) is further operated to couple with pinion (307) such that it moves distally in correlation with first rack (302) translating proximally as discussed above. Similarly, as second rack (303) translates proximally to rotate second pinion (308), a vacuum inlet spool valve plug (320) (see FIGS. 13A-C) from vacuum source (228) is opened to thereby couple vacuum source (228) with shaft assembly (212). Vacuum projection (316) is further operated to couple with pinion (308) such that it moves distally in correlation with second rack (303) translating proximally as discussed above. In accordance with these features, operation of pull cables (244, 246) by instrument base (216) thereby operates spool valve assembly (226). As shown in the present example, vacuum and fluid inlet spool valve plugs (320, 318) are movably positioned within valve body (300) to a number of predetermined positions described in greater detail below for desired flow of fluid.

Depicted in FIGS. 13A-13C are three different valve position combinations of spool valve assembly (226). Shown in FIG. 13A is spool valve assembly (226) in a first configuration whereby fluid and vacuum inlet spool valve plugs (318, 320), each respectively being coupled with vacuum source (228) or fluid source (230), are both in a closed position. Springs (322, 324) act to bias spool valve plugs (318, 320) and racks (302, 303) in proximal positions until pull cables (244, 246,) selectively pull one or both racks (302, 303) distally using valve adapter (224) as described above. In the closed positions, vacuum source (228) and fluid source (230) are each fluidly decoupled, which may also be referred to herein as fluidly disconnected, from shaft assembly (212) and distal opening (215) of end effector (214). More particularly, outlet opening (306) of spool valve assembly (226) and valve adapter (224) are disconnected from both vacuum and fluid inlet openings (304, 305). Shown in FIG. 13B is spool valve assembly (226) in a second configuration whereby fluid inlet spool valve plug (318) is in an open position to fluidly couple fluid source (230) and fluid inlet opening (305) with outlet opening (306) for fluid communication therethrough while vacuum inlet spool valve plug (320) remains in the closed position. Shown in FIG. 13C is spool valve assembly (226) in a third configuration whereby vacuum inlet spool valve plug (320) is in an open position to (318) fluidly couple vacuum source (228) and vacuum inlet opening (304) with outlet opening (306) for fluid communication therethrough while fluid inlet spool valve plug (318) remains in the closed position.

### III. Exemplary Stopcock Valve Assemblies for a Suction-Irrigation Surgical Instrument

In some examples it may be desirable to incorporate certain alternative stopcock valve assemblies similar in some respects to spool valve assembly (226) into instrument (210) described above or other suitable instruments. For instance, in some examples it may be desirable to include certain features to directly rotatably drive alternative stopcock valve assemblies rather than convert such rotation to translation for driving spool valve assembly (226) as discussed above in order to rotatably drive such stopcock valve assemblies with less complexity and greater performance.

### A. Horizontal Stopcock Valve Assembly

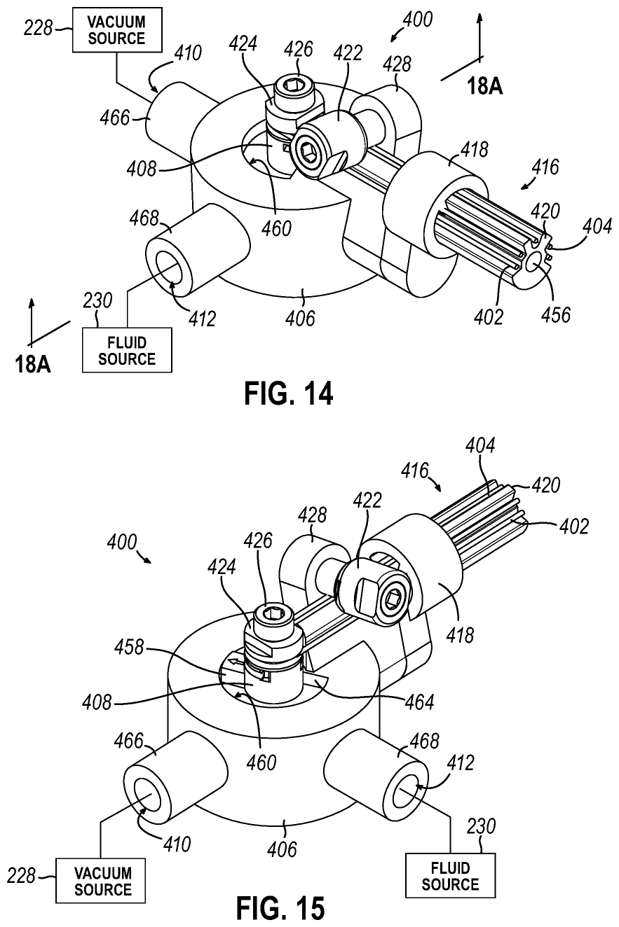

Depicted in FIGS. 14-16 is a horizontal stopcock valve assembly (400) for operating in place of one or more portions of valve adapter (224) and spool valve assembly (226). Similar to spool valve assembly (226), horizontal stopcock valve assembly (400) is configured to receive one or more inputs, for example, vacuum source (228) and fluid source (230), and selectively output various combinations of the one or more inputs. Specifically, horizontal stopcock valve assembly (400) allows for one rotary input mechanism to drive the multi-position horizontal stopcock valve assembly (400) and allow for the multiple output states. Rotary input mechanism of the present example is provided at least in part in the form of pull cables (402, 404). Pull cables (402, 404) are coupled at one end to an instrument base, such as instrument base (216), which translates pull cables (402, 404) distally and proximally to operate horizontal stopcock valve assembly (400) similar to valve adapter (224) and valve assembly (226) described above.

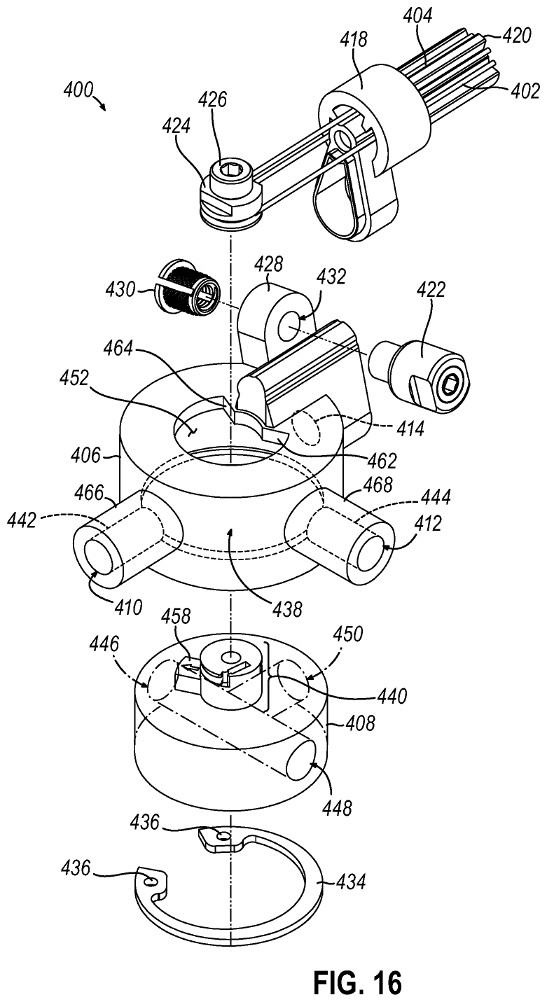

As shown, horizontal stopcock valve assembly (400) includes a valve body (406) and a valve plug (408), where valve plug (408) is configured to axially rotate relative to valve body (406). Valve body (406) includes a vacuum inlet opening (410) and a fluid inlet opening (412) in extension arms (466, 468) for receiving the one or more inputs to transfer to valve plug (408) when valve plug (408) is rotated to certain positions. Valve body (406) further includes an outlet opening (414) (see FIG. 16). To accept valve driving inputs from pull cables (402, 404), a drive assembly (416) of valve assembly (400) in the present example includes a cap (418) to stabilize the positioning of pull cables (402, 404) relative to shaft assembly (420), a capstan (422), and a pulley (424) are rotatably coupled with valve body (406) using a connector (426), which in some instances may be a threaded connector. As best shown in FIG. 16, capstan (422) may couple with an arm (428) of valve body (406) via an expandable threaded connector (430) so it may be securely held into place within opening (432) of arm (428). Expandable threaded connector (430) may be, for example, a screw-to-expand insert. Valve assembly (400) of the present example also includes a retaining ring (434) to retain valve plug (408) within valve body (406) via connector openings (436).

To assemble valve assembly (400), valve plug (408) is inserted into valve body (406) through a lower opening (438) of valve body (406) and secured into place using retaining ring (434). Next, upper portion (440) of valve plug (408) is coupled with pulley (424) of drive assembly (416) using connector (426), and capstan (422) is coupled with arm (428) of valve body (406) using connector (430).

As best shown in FIG. 16, vacuum inlet opening (410) and fluid inlet opening (412) of valve body (406) are each respectively coupled with horizontal lumens (442, 444) within valve body (406). Horizontal lumens (442, 444) are each configured to selectively fluidly couple with first, second, and third valve conduits (446, 448, 450) of valve plug (408) as valve plug (408) with associated valve conduits (446, 448, 450) rotates about multiple positions. Valve conduits (446, 448, 450) of the present example are collectively in fluid communication with each other and, as shown, define a T-shape such that first, second, and third valve conduits (446, 448, 450) collectively define a common conduit in common fluid communication although the invention is not intended to be unnecessarily limited to such shape on commonality as shown in the present example. Valve conduits (446, 448, 450) are configured to align and fluidly communicate with vacuum and fluid inlet openings (410, 412) and outlet opening (414) for transferring fluid (e.g., suction air or irrigation fluids) therethrough in a variety of positions as discussed below in greater detail. Valve plug (408) and an interior surface (452) of valve body (406) are in intimate contact that inhibits fluid leakage between valve plug (408) and valve body (406).

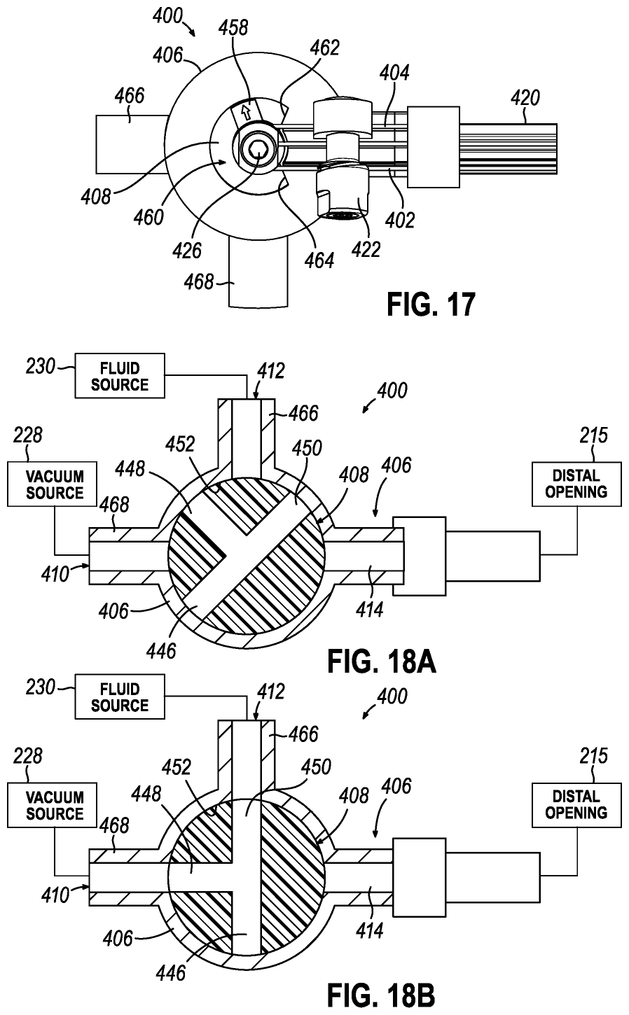

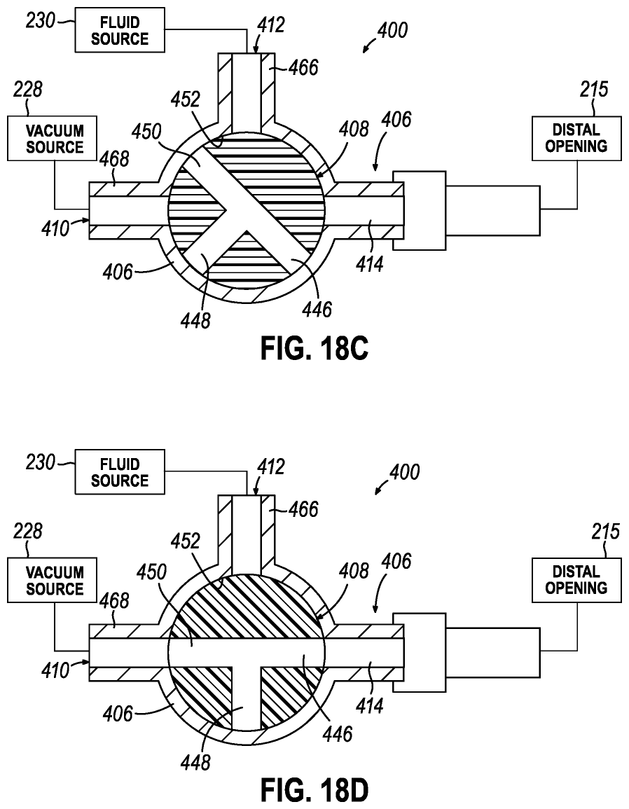

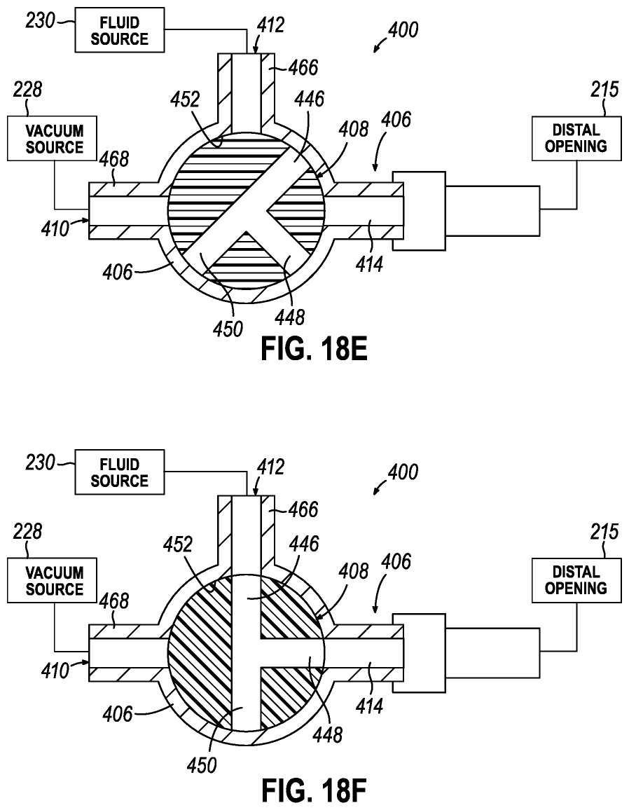

As depicted in FIG. 17, top surface of valve plug (408) includes a rotation stop arm (458). As valve plug (408) is rotated relative to valve body (406), rotational stop arm (458) is configured such that it moves within a channel (460) between stop surfaces (462, 464). As such, valve plug (408) is thereby selectively rotatable about 225 degrees relative to valve body (406) between the various valve positions which are approximately equidistance apart and will be described in greater detail below in FIGS. 18A-F. In one example, one or more stop surfaces (462, 464) may provide a homing position from which to base further positioning for robotic control thereof.

In use, as depicted in FIG. 18A, valve plug (408) of horizontal stopcock valve assembly (400) may be rotated, such as by pulleys (402, 404) via drive assembly (416), to a first position such that interior surface (452) blocks fluid communication at each of first, second, and third conduits (446, 448, 450) and neither vacuum source (228) with vacuum inlet opening (410) nor fluid source (230) with fluid inlet opening (412) are fluidly connected with outlet opening (414). Thus, neither vacuum source (228) nor fluid source (230) is fluidly coupled with shaft assembly (420) and distal opening (215) of end effector (214) (see FIG. 7).

Rotating valve plug (408) counterclockwise from the first position to a second position shown in FIG. 18B fluidly connects first conduit (446) and second conduit respectively to vacuum inlet opening (410) and fluid inlet opening (412) while third conduit (450) and outlet opening (414) remain blocked. Thus, vacuum source (228) draws upon fluid source (230) to actively prime instrument (210) (see FIG. 7) with irrigation fluid without directing any such irrigation fluid to shaft assembly (420) and distal opening (215) of end effector (214) (see FIG. 7).

Rotating valve plug (408) counterclockwise from the second position to a third position shown in FIG. 18C again blocks fluid communication at each of first, second, and third conduits (446, 448, 450) with interior surface (452) such that neither vacuum source (228) with vacuum inlet opening (410) nor fluid source (230) with fluid inlet opening (412) are fluidly connected with outlet opening (414). Thus, neither vacuum source (228) nor fluid source (230) is fluidly coupled with shaft assembly (420) and distal opening (215) of end effector (214) (see FIG. 7) in the third position.

Rotating valve plug (408) counterclockwise from the third position to a fourth position shown in FIG. 18D fluidly connects first conduit (446) and third conduit (450) respectively to vacuum inlet opening (410) and outlet opening (414) while second conduit (448) and fluid source (230) are blocked. Thus, vacuum source (228) draws upon fluid and any associated debris at distal opening (215) via shaft assembly (420) (see FIG. 7) without application of the irrigation fluid from fluid source (230).

Rotating valve plug (408) counterclockwise from the fourth position to a fifth position shown in FIG. 18E again blocks fluid communication at each of first, second, and third conduits (446, 448, 450) with interior surface (452) such that neither vacuum source (228) with vacuum inlet opening (410) nor fluid source (230) with fluid inlet opening (412) are fluidly connected with outlet opening (414). Thus, neither vacuum source (228) nor fluid source (230) is fluidly coupled with shaft assembly (420) and distal opening (215) of end effector (214) (see FIG. 7) in the fifth position.

Rotating valve plug (408) counterclockwise from the fifth position to a sixth position shown in FIG. 18F fluidly connects first conduit (446) and second conduit (448) respectively to fluid inlet opening (412) and outlet opening (414) while third conduit (450) and vacuum source (228) are blocked. Thus, fluid source (230) directs the irrigation fluid to distal opening (215) via shaft assembly (420) (see FIG. 7) without application of the vacuum from vacuum source (228).

In the present examples, first, second, third, fourth, fifth, and sixth positions with the above associated states of functionality are discretely set along a single rotational direction. The invention is not intended to be unnecessarily limited to only successive applications of the first, second, third, fourth, fifth, and sixth positions and, instead, may be selected as desired by directly rotating valve plug (408) without added translation via at least one rotary drive input, such as at least one of pinions (248, 250) (see FIG. 10). Moreover, to the extent that conduits (446, 448, 450) are arranged relative to inlet openings (410, 412) and/or outlet opening (414) so as to inhibit fluid communication therethrough, such conduits (446, 448, 450) may also be referred to as fluidly disconnected from inlet openings (410, 412) and/or outlet opening (414) as applicable.

### B. Axial Stopcock Valve Assembly

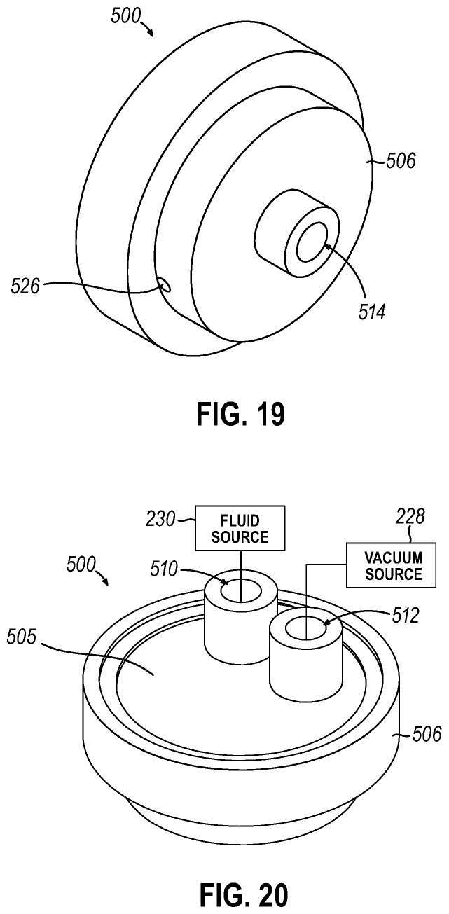

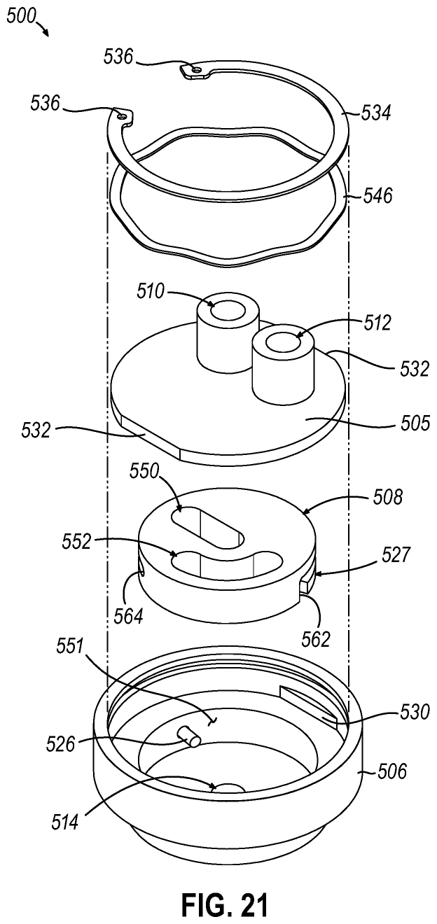

Depicted in FIGS. 19-21 is an axial stopcock valve assembly (500) for operating in place of the combinations of valve adapter (224) and spool valve assembly (226). Similar to spool valve assembly (226) and horizontal stopcock valve assembly (400), axial valve assembly (500) is configured to receive one or more inputs, for example, vacuum source (228) and fluid source (230), and selectively output various combinations of the one or more inputs. Specifically, axial stopcock valve assembly (500) allows for one rotary input mechanism to drive the multi-position axial stopcock valve assembly (500) and allow for the multiple output states. Rotary input mechanism of the present example is provided at least in part in the form of a drive assembly having pull cables, such as drive assembly (416) and pull cables (402, 404) and/or at least one of pinions (248, 250) (see FIG. 10) described above. Pull cables (402, 404) are coupled at one end to an instrument base, such as instrument base (216), which translates pull cables (402, 404) distally and proximally to operate axial stopcock valve assembly (500) similar to valve adapter (224) and valve assembly (226) described above.

As shown, axial stopcock valve assembly (500) includes a valve body (506) and a valve plug (508). Valve body (506) of the present example also includes a valve cap (505) separable from a remainder of valve body (506), where valve plug (508) is configured to axially rotate relative to valve cap (505). Valve cap (505) includes a fluid inlet opening (510) and a vacuum inlet opening (512) for receiving the one or more inputs to transfer to valve plug (508) when valve plug (508) is rotated to certain positions. Valve body (506) further includes an outlet opening (514) configured to fluidly couple with shaft assembly (212) and distal opening (215) of end effector (214) (see FIG. 7). To accept valve driving inputs from pull cables (402, 404) to thereby axially rotate valve plug (508) relative to valve body (506), including valve cap (505), valve plug (508) includes an engagement portion (not shown), whereas valve body (506) includes an access portion (not shown). By way of example, inputs from pull cables (402, 404) are configured to operatively connect with engagement portion (not shown) on valve plug (508) through access portion (not shown) of valve body (506) for coupling with a drive assembly, such as drive assembly (416), for directly and selectively driving rotation of valve plug (508) relative to valve body (506). Valve assembly (500) also includes a retaining ring (534) to retain valve cap (505) and valve plug (508) to a remainder of valve body (506) via connector openings (536).

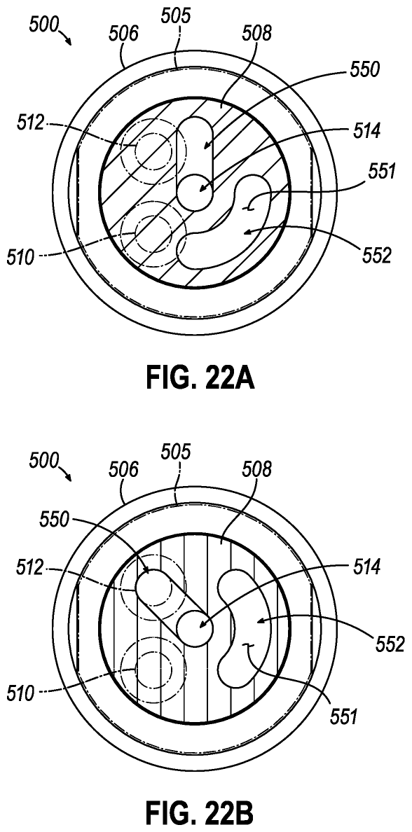

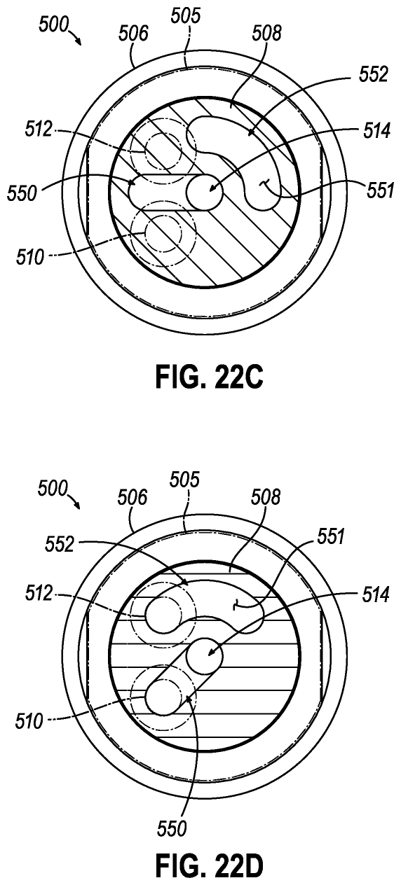

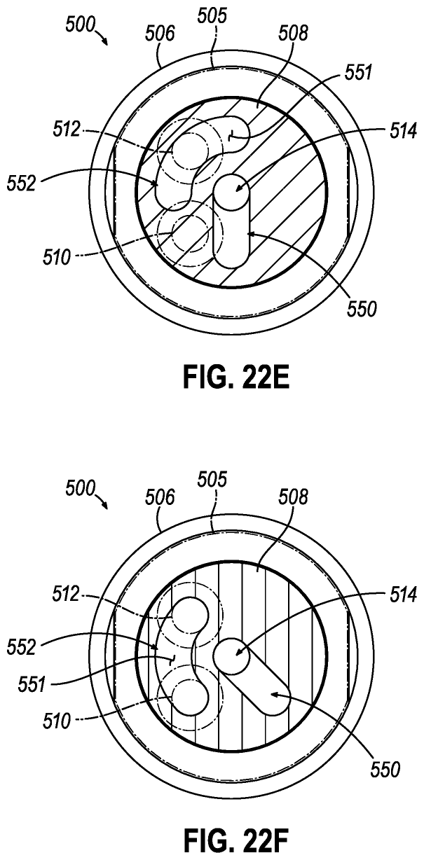

Valve body (506) has a central cavity which accepts valve cap (505) and valve plug (508). Valve plug (508) and an interior surface (551) of valve body (506) are in intimate contact that inhibits fluid leakage between valve plug (508) and valve body (506). A base of valve body (506) includes a rotation limiting feature (526), such as a pin, protruding from interior surface (551) of valve body (506) so as to interact and translate within a corresponding channel (527) formed on a side exterior surface of valve plug (508) and defining stop surfaces (562, 564) on channel ends thereof. As such, valve plug (508) is only permitted to axially rotate about 220 degrees relative to valve body (506) between the various valve positions which are approximately equidistance apart and will be described in greater detail below in FIGS. 22A-F. In one example, one or more stop surfaces (562, 564) may provide a homing position from which to base further positioning for robotic control thereof.

Valve body (506) further includes protruding features (530) to interact with corresponding flat features (532) of valve cap (505) to thereby secure valve cap (505) in a fixed rotational position relative to protruding features (530) of valve body (506). Also in the present example, a wave spring (546) may be provided between retaining ring (534) and valve cap (505) to sandwich valve plug (508) in compression between valve cap (505) and a remainder of valve body (506) for fluid sealing while allowing for rotation of valve plug (508) during use.

Valve plug (508) further includes first and second conduits (550, 552) which are positioned, shaped, and sized to selectively align with fluid and vacuum inlet openings (510, 512) to provide fluid pathways through valve plug (508) between vacuum source (228), fluid source (230), and outlet opening (514) toward shaft assembly and distal opening (215) of end effector (214) (see FIG. 7). As will be described in greater detail below, first conduit (550) is generally linear in a plane perpendicular to a central rotational axis of valve plug (580), whereas second conduit (552) is generally arcuate in the plane perpendicular to the central rotational axis of valve plug (580). In this respect, first conduit (550) is configured to extend from fluid connection with outlet opening (514) selectively to each of fluid and vacuum inlet openings (510, 512) for fluid communication therebetween. In contrast, second conduit (552) is configured to extend between each of fluid and vacuum inlet openings (510, 512) for selective fluid communication therebetween.

In use, as depicted in FIG. 22A, valve plug (508) of axial stopcock valve assembly (500) may be rotated, such as by pulleys (402, 404) via drive assembly (416), to a first position such that interior surface (551) blocks fluid communication at each of first and second conduits (550, 552) and neither vacuum source (228) with vacuum inlet opening (510) nor fluid source (230) with fluid inlet opening (512) are fluidly connected with outlet opening (514). Thus, neither vacuum source (228) nor fluid source (230) is fluidly coupled with shaft assembly (420) and distal opening (215) of end effector (214) (see FIG. 7).

Rotating valve plug (508) counterclockwise from the first position to a second position shown in FIG. 22B fluidly connects first conduit (550) between vacuum inlet opening (512) and outlet opening (514) while second conduit (552) and fluid source (230) are blocked. Thus, vacuum source (228) draws upon fluid and any associated debris at distal opening (215) via shaft assembly (420) (see FIG. 7) without application of the irrigation fluid from fluid source (230).

Rotating valve plug (508) counterclockwise from the second position to a third position shown in FIG. 22C again blocks fluid communication at each of first and second conduits (550, 552) with interior surface (551) such that neither vacuum source (228) with vacuum inlet opening (512) nor fluid source (230) with fluid inlet opening (510) are fluidly connected with outlet opening (514). Thus, neither vacuum source (228) nor fluid source (230) is fluidly coupled with shaft assembly (420) and distal opening (215) of end effector (214) (see FIG. 7) in the third position.

Rotating valve plug (508) counterclockwise from the third position to a fourth position shown in FIG. 22D fluidly connects first conduit (550) between fluid inlet opening (510) and outlet opening (514) while second conduit (552) and vacuum source (228) are blocked. In the present example, a portion of second conduit (552) fluidly connects to vacuum inlet opening (512), but no other portion of second conduit (552) fluidly connects to another outlet such that no flow of vacuum occurs therethrough. Thus, fluid source (230) directs the irrigation fluid to distal opening (215) via shaft assembly (420) (see FIG. 7) without application of the vacuum from vacuum source (228).

Rotating valve plug (508) counterclockwise from the fourth position to a fifth position shown in FIG. 22E again blocks fluid communication at each of first and second conduits (550, 552) with interior surface (551) such that neither vacuum source (228) with vacuum inlet opening (512) nor fluid source (230) with fluid inlet opening (510) are fluidly connected with outlet opening (514). Thus, neither vacuum source (228) nor fluid source (230) is fluidly coupled with shaft assembly (420) and distal opening (215) of end effector (214) (see FIG. 7) in the fifth position.

Rotating valve plug (508) counterclockwise from the fifth position to a sixth position shown in FIG. 22F fluidly connects second conduit (552) between vacuum inlet opening (512) and fluid inlet opening (510) while outlet opening (514) is blocked. In the present example, a portion of first conduit (550) remains fluidly connected outlet opening (514), but no other portion of first conduit (550) fluidly connects to another inlet such that no flow through outlet opening (514) occurs. Thus, vacuum source (228) draws upon fluid source (230) to actively prime instrument (210) (see FIG. 7) with irrigation fluid without directing any such irrigation fluid to shaft assembly (420) and distal opening (215) of end effector (214) (see FIG. 7).

In the present examples, first, second, third, fourth, fifth, and sixth positions with the above associated states of functionality are discretely set along a single rotational direction. The invention is not intended to be unnecessarily limited to only successive applications of the first, second, third, fourth, fifth, and sixth positions and, instead, may be selected as desired by directly rotating valve plug (508) without added translation via at least one rotary drive input, such as at least one of pinions (248, 250) (see FIG. 10). Moreover, to the extent that conduits (550, 552) are arranged relative to inlet openings (510, 512) and/or outlet opening (514) so as to inhibit fluid communication therethrough, such conduits (550, 552) may also be referred to as fluidly disconnected from inlet openings (510, 512) and/or outlet opening (514) as applicable.

### C. Vertical Stopcock Valve Assembly

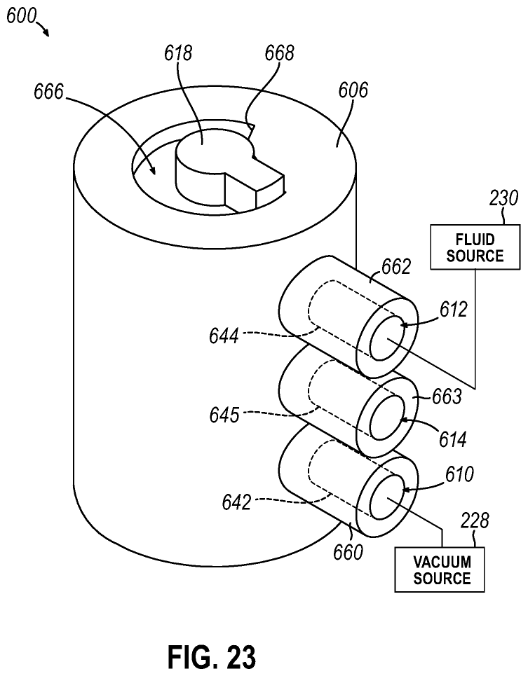

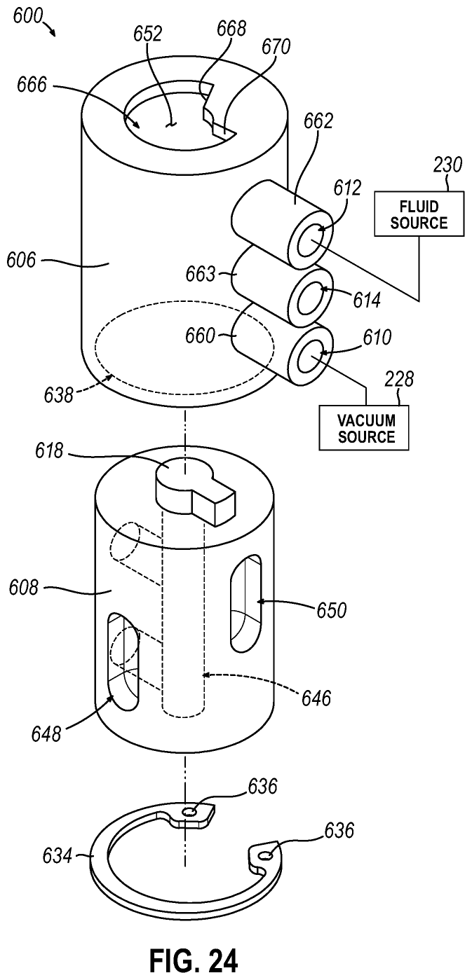

Depicted in FIGS. 23-24 is a vertical stopcock valve assembly (600) for operating in place of the combinations of valve adapter (224) and spool valve assembly (226). Similar to spool valve assembly (226), horizontal stopcock valve assembly (400), and axial stopcock valve assembly (500), vertical stopcock valve assembly (600) is configured to receive one or more inputs, for example, vacuum source (228) and fluid source (230), and selectively output various combinations of the one or more inputs. Specifically, vertical stopcock valve assembly (600) allows for one rotary input mechanism to drive the multi-position vertical stopcock valve assembly (600) and allow for the multiple output states. Rotary input mechanism of the present example is provided at least in part in the form of a drive assembly having pull cables, such as drive assembly (416) and pull cables (402, 404) and/or at least one of pinions (248, 250) (see FIG. 10) described above. Pull cables (402, 404) may be coupled at one end to an instrument base, such as instrument base (216), which translates pull cables (402, 404) distally and proximally to operate vertical stopcock valve assembly (600) similar to valve adapter (224) and valve assembly (226) described above.

As shown, vertical stopcock valve assembly (600) includes a valve body (606) and a valve plug (608), where valve plug (608) is configured to axially rotate relative to valve body (606). Valve body (606) includes a vacuum inlet opening (610) and a fluid inlet opening (612) in extension arms (660, 662) for receiving the one or more inputs to transfer to valve plug (608) when valve plug (608) is rotated to certain positions. Valve body (606) further includes an outlet opening (614) in another extension arm (663). To accept valve driving inputs from pull cables (402, 404) to thereby axially rotate valve plug (608) relative to valve body (606), valve plug (608) includes a driven portion (618) for coupling with a drive assembly, such as drive assembly (416). Valve assembly (600) also includes a retaining ring (634) to retain valve plug (608) within valve body (606) via connector openings (636).

To assemble valve assembly (600), valve plug (608) is inserted into valve body (606) through a lower opening (638) of valve body (606) and secured into place using retaining ring (634). Driven portion (618) of valve (608) is coupled with a drive assembly.

Vacuum inlet opening (610) and fluid inlet opening (612) of valve body (606) are each respectively coupled with horizontal lumens (642, 644) within valve body (606) (see FIG. 23) for selectively fluidly coupling with valve plug (608) for providing various combinations of fluid connectivity between vacuum source (228) and fluid source (230) through horizontal lumen (645) to outlet opening (614) as valve plug (608) rotates about multiple positions. To this end, valve plug (608) includes a first conduit (646), a second conduit (648), and a third conduit (650). First conduit (646) is configured to selectively fluidly connect vacuum and fluid inlet openings (610, 612) and extends from an outer radial surface and along a central rotational axis of valve plug (608) for extending between vacuum and fluid inlet openings (610, 612). Second conduit (648) is configured to selectively fluidly connect vacuum inlet opening (610) to outlet opening (614) and extends as a blind hole radially into outer radial surface of valve plug (608) for extending between vacuum inlet opening (610) to outlet opening (614). Third conduit (650) is configured to selectively fluidly connect fluid inlet opening (612) to outlet opening (614) and extends as a blind hole radially into outer radial surface of valve plug (608) for extending between fluid inlet opening (612) to outlet opening (614). As such, outlet opening (614) is configured to selectively and fluidly connect with and transfer fluid (e.g., suction air or irrigation fluids) associated with vacuum source (228) and fluid source (230). Valve plug (608) and an interior surface (652) of valve body (606) are in intimate contact that inhibits fluid leakage between valve plug (608) and valve body (606).

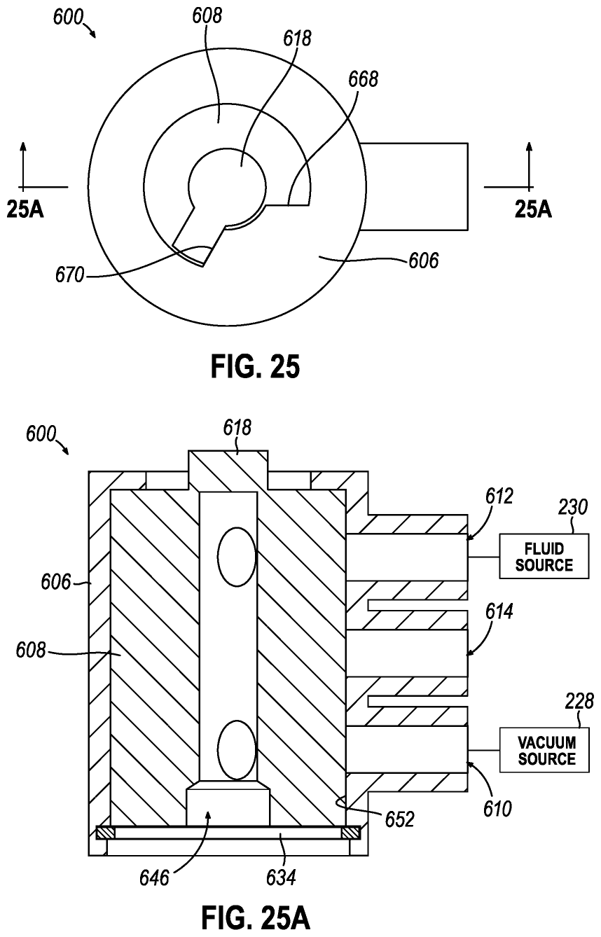

As depicted in FIG. 24, top surface of valve (608) includes driven portion (618) which also functions as a rotation stop feature. As valve plug (608) is rotated relative to valve body (606), driven portion (618) is configured such that it moves within a channel (666) between stop surfaces (668, 670). As such, valve plug (608) is thereby selectively rotatable about 240 degrees relative to valve body (606) between the various valve positions which are approximately equidistance apart and will be described in greater detail below in FIGS. 25-30A. In one example, one or more stop surfaces (668, 670) may provide a homing position from which to base further positioning for robotic control thereof.

In use, as depicted in FIGS. 25-25A, valve plug (608) of vertical stopcock valve assembly (600) may be rotated, such as by pulleys (402, 404) via drive assembly (416), to a first position such that interior surface (652) blocks fluid communication at each of first, second, and third conduits (646, 648, 650) and neither vacuum source (228) with vacuum inlet opening (610) nor fluid source (230) with fluid inlet opening (612) are fluidly connected with outlet opening (614). Thus, neither vacuum source (228) nor fluid source (230) is fluidly coupled with shaft assembly (420) and distal opening (215) of end effector (214) (see FIG. 7).

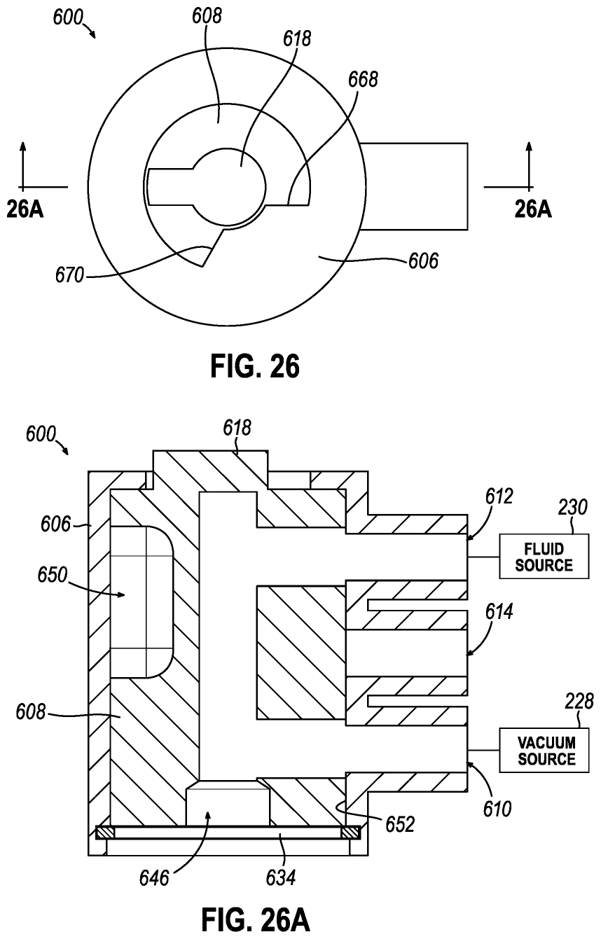

Rotating valve plug (608) clockwise from the first position to a second position shown in FIGS. 26-26A fluidly connects first conduit (646) between vacuum inlet opening (610) and fluid inlet opening (612) while second conduit (648), third conduit (650), and outlet opening (614) remain blocked. Thus, vacuum source (228) draws upon fluid source (230) to actively prime instrument (210) (see FIG. 7) with irrigation fluid without directing any such irrigation fluid to shaft assembly (420) and distal opening (215) of end effector (214) (see FIG. 7).

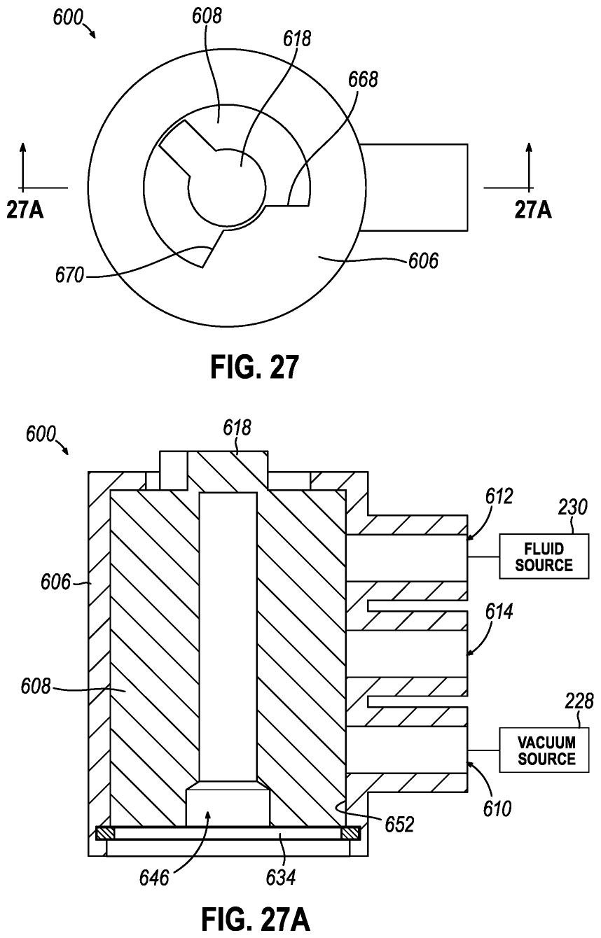

Rotating valve plug (608) clockwise from the second position to a third position shown in FIGS. 27-27A again blocks fluid communication at each of first, second, and third conduits (646, 648, 650) with interior surface (652) such that neither vacuum source (228) with vacuum inlet opening (610) nor fluid source (230) with fluid inlet opening (612) are fluidly connected with outlet opening (614). Thus, neither vacuum source (228) nor fluid source (230) is fluidly coupled with shaft assembly (420) and distal opening (215) of end effector (214) (see FIG. 7) in the third position.

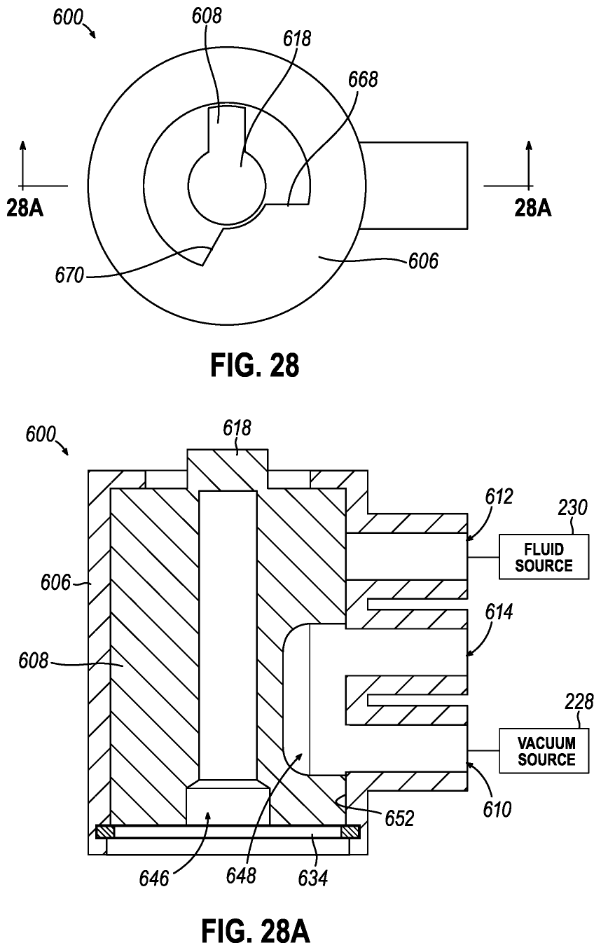

Rotating valve plug (608) clockwise from the third position to a fourth position shown in FIGS. 28-28A fluidly connects second conduit (648) between vacuum inlet opening (610) and outlet opening (614) while first conduit (646), third conduit, (650), and fluid source (230) are blocked. Thus, vacuum source (228) draws upon fluid and any associated debris at distal opening (215) via shaft assembly (420) (see FIG. 7) without application of the irrigation fluid from fluid source (230).

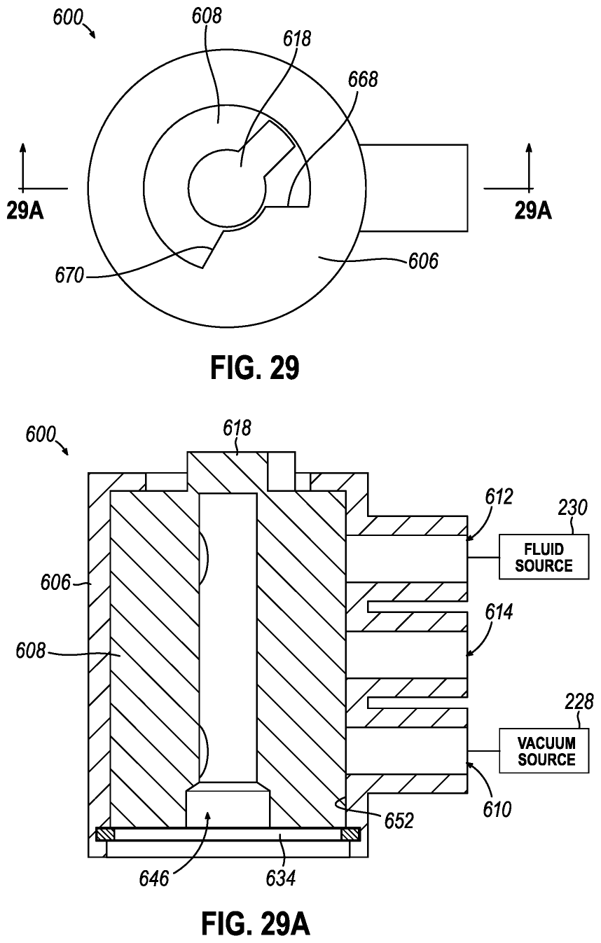

Rotating valve plug (608) clockwise from the fourth position to a fifth position shown in FIGS. 29-29A again blocks fluid communication at each of first, second, and third conduits (646, 648, 650) with interior surface (652) such that neither vacuum source (228) with vacuum inlet opening (610) nor fluid source (230) with fluid inlet opening (612) are fluidly connected with outlet opening (614). Thus, neither vacuum source (228) nor fluid source (230) is fluidly coupled with shaft assembly (420) and distal opening (215) of end effector (214) (see FIG. 7) in the fifth position.

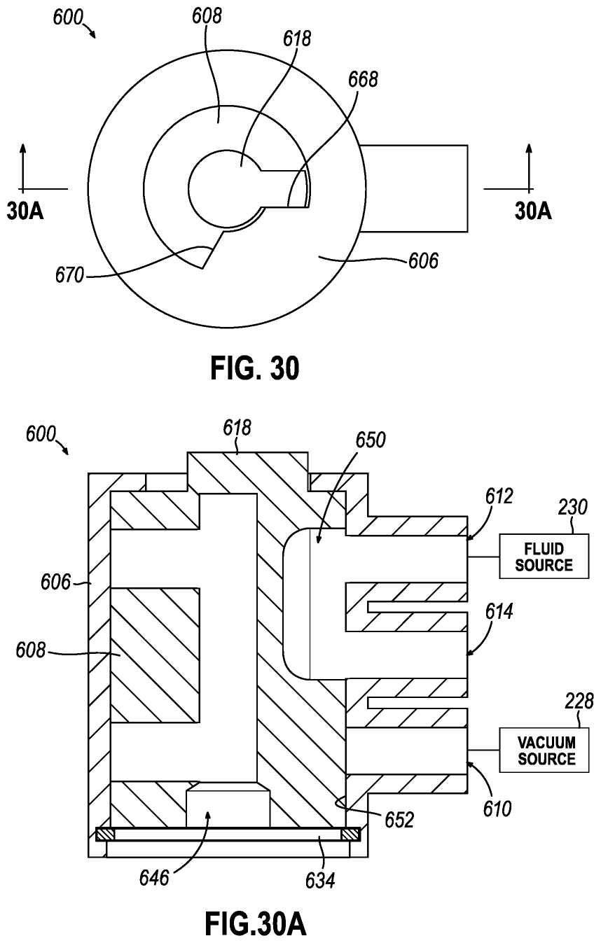

Rotating valve plug (408) clockwise from the fifth position to a sixth position shown in FIGS. 30-30A fluidly connects third conduit (650) between fluid inlet opening (612) and outlet opening (614) while first conduit (646), second conduit (648), and vacuum source (228) are blocked. Thus, fluid source (230) directs the irrigation fluid to distal opening (215) via shaft assembly (420) (see FIG. 7) without application of the vacuum from vacuum source (228).

In the present examples, first, second, third, fourth, fifth, and sixth positions with the above associated states of functionality are discretely set along a single rotational direction. The invention is not intended to be unnecessarily limited to only successive applications of the first, second, third, fourth, fifth, and sixth positions and, instead, may be selected as desired by directly rotating valve plug (608) without added translation via at least one rotary drive input, such as at least one of pinions (248, 250) (see FIG. 10). Moreover, to the extent that conduits (646, 648, 650) are arranged relative to inlet openings (610, 612) and/or outlet opening (614) so as to inhibit fluid communication therethrough, such conduits (646, 648, 650) may also be referred to as fluidly disconnected from inlet openings (610, 612) and/or outlet opening (614) as applicable.

### IV. Suction-Irrigation Surgical Instrument with Disposable Valve Assemblies

In some examples it may be desirable to incorporate certain alternative valve assemblies into instrument (210) or other suitable instruments similar to valve assemblies (226, 400, 500, 600) described above. For instance, in some examples it may be desirable install and/or remove a disposable valve assembly relative to a reusable portion of instrument (210). Such disposable portions may be desirable to promote ease of use, simplify cleaning procedures, and reduce costs of the overall instrument (210) per use.

### A. A First Example of a Disposable Valve Assembly

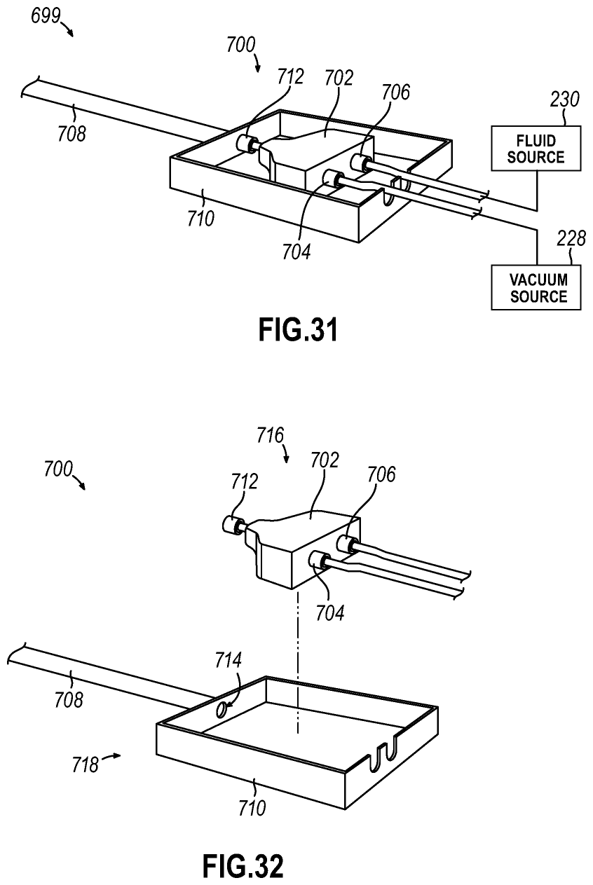

Depicted in FIG. 31 is a third exemplary surgical instrument (699) having a first example of a reusable instrument housing (710) with a shaft assembly (708) and a first example of a disposable valve assembly (700) having a valve body (702) configured to receive first and second fluid ports (704, 706) for coupling with one or more medical treatment devices (e.g., a vacuum source (228) and a fluid source (230)). Valve body (702) is further configured to couple with shaft assembly (708) to selectively provide fluid communication between end effector (not shown) of an instrument (699). By way of example, valve assembly (700) may be configured similar to, and operable to, any of valve assemblies (226, 400, 500, 600), although the invention is not intended to be unnecessarily limited to such valve assemblies (226, 400, 500, 600). In some versions, valve assembly (700) may be secured within instrument housing (710) such that outlet port (712) of valve assembly (700) may fluidly couple with shaft assembly (708) through an opening (714) through an exterior surface of instrument housing (710). In some versions, each of ports (704, 706, 712) can include secure fittings such as, for example, luer fittings or any other similar connector operable to provide a leak-free fluid coupling.

Depicted in FIG. 32 is disposable valve assembly (700) shown disassembled. Prior to performing a surgical operation with disposable valve assembly (700), an operator may accordingly assemble a disposable portion (716) and a reusable portion (718). Disposable portion (716) may include valve body (702), and any internal valve components, and ports (704, 706, 712). Reusable portion (718) may include all other components of surgical instrument (699), such as shaft assembly (708), instrument housing (710), end effector (not shown), and any drive components operable to manipulate valve assembly (700). After use, disposable portion (716) may be thrown away as opposed to being cleaned and sterilized for later use, while reusable portion (718) may be cleaned and sterilized for later use.

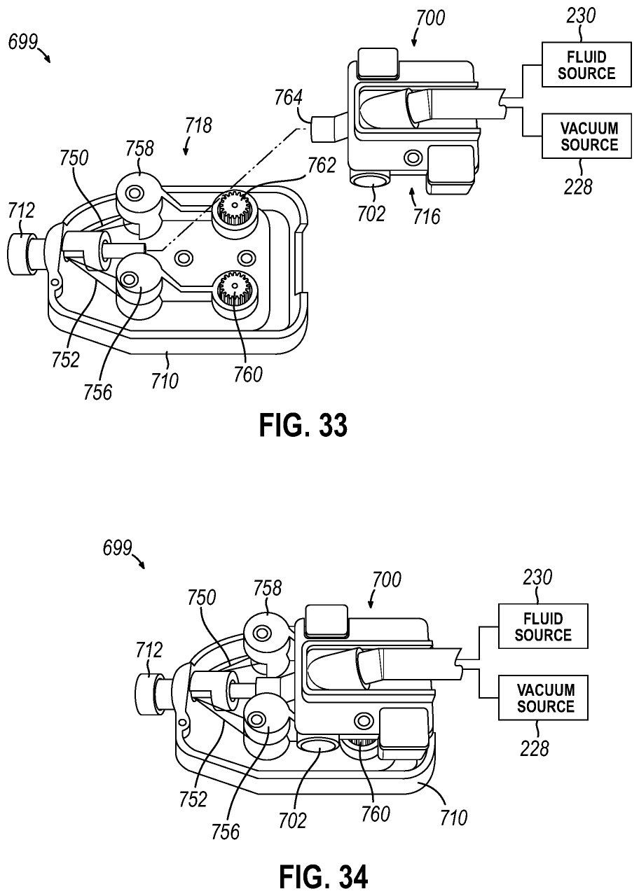

Depicted in FIGS. 33-34 is one exemplary embodiment of valve assembly (700). Particularly, in FIG. 33, disposable portion (716) is shown removed from reusable portion (718), further shown with an upper portion of the housing of valve body (702) removed. As described above, valve assembly (700) may be configured to operatively couple with one or more pull cables (750, 752) to direct operation of valve assembly (700). Pull cables (750, 752) may be directed around pulleys (756, 758) to rotate pinions (760, 762), respectively. Pinions (760, 762) operate to direct internal movements of valve assembly (700). Valve assembly (700) may be configured and operable similar to any of valve assemblies (226, 400, 500, 600) described herein. To couple disposable portion (716) with reusable portion (718), disposable portion (716) is positioned over pinions (760, 762) to operatively couple with pinions (760, 762), and an outlet opening (764) is fluidly coupled with an input (766) of reusable portion (718).

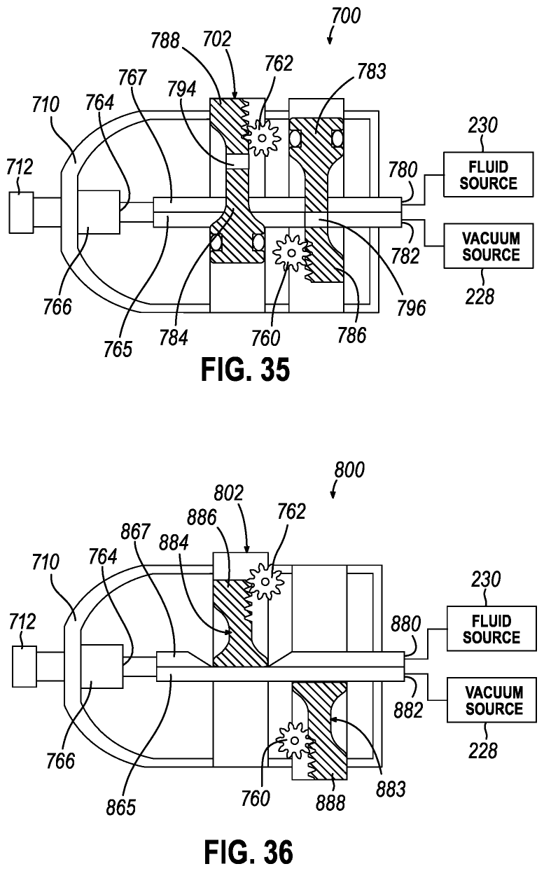

By way of example, FIG. 35 depicts aspects of valve assembly (700) more particularly as an example of a trumpet valve assembly (700). Specifically, trumpet valve assembly (700) selectively fluidly connects vacuum source (228) and fluid source (230) with outlet port (712). Trumpet valve assembly (700) includes outlet opening (764), valve body (702), a fluid inlet opening (780), and a vacuum inlet opening (782). A vacuum inlet trumpet valve plug (783) is configured to selectively fluidly connect vacuum inlet opening (782) to outlet opening (764) via a vacuum body conduit (765), whereas a fluid inlet trumpet valve plug (784) is configured to selectively fluidly connect fluid inlet opening (780) to outlet opening (764) via a fluid body conduit (767). As shown in the present example, fluid and vacuum inlet trumpet valve plugs (783, 784) have respective racks (786, 788) configured to be selectively translated by pinions (762, 760) to align a fluid plug conduit (794) with fluid body conduit (767) and a vacuum plug conduit (796) with a vacuum body conduit (765) as desired for fluid communication therethrough. Thus, trumpet valve assembly (700) is configured to selectively fluidly connect vacuum source (228) and fluid source (230) to shaft assembly (708) (see FIG. 31) and may be used for directing fluid flow as discussed above with respect to one or more of valve assemblies (226, 400, 500, 600).

By way of further example, FIG. 36 depicts an alternative disposable valve assembly (800) for use in place of trumpet valve assembly (700) discussed above. The present example more particularly shows alternative disposable valve assembly (800) as a pinch valve assembly (800) with like numbers indicating like features discussed above in greater detail. Specifically, pinch valve assembly (800) selectively fluidly connects vacuum source (228) and fluid source (230) with outlet port (712). Pinch valve assembly (800) includes outlet opening (764), a valve body (802), a fluid inlet opening (880), and a vacuum inlet opening (882). A vacuum inlet pinch valve plug (883) is configured to selectively fluidly connect vacuum inlet opening (882) to outlet opening (764) via a vacuum body conduit (865), whereas a fluid inlet pinch valve plug (884) is configured to selectively fluidly connect fluid inlet opening (880) to outlet opening (764) via a fluid body conduit (867). As shown in the present example, fluid and vacuum inlet pinch valve plugs (883, 884) have respective racks (886, 888) configured to be selectively translated by pinions (762, 760) to pinch or release fluid and vacuum conduits (867, 865) for allowing or inhibiting fluid communication therethrough as desired. Thus, pinch valve assembly (800) is configured to selectively fluidly connect vacuum source (228) and fluid source (230) to shaft assembly (708) (see FIG. 31) and may be used for directing fluid flow as discussed above with respect to one or more of valve assemblies (226, 400, 500, 600).

### B. A Second Example of a Disposable Valve Assembly

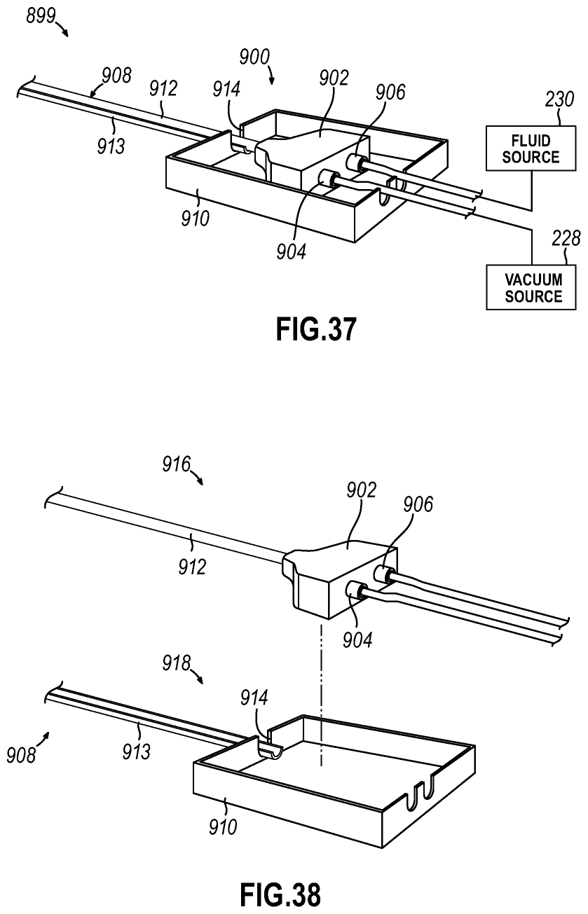

Depicted in FIG. 37 is a fourth exemplary surgical instrument (899) having a second example of a reusable instrument housing (910) with a shaft assembly (908) and a second example of a disposable valve assembly (900) having a valve body (902) configured to receive first and second fluid ports (904, 906) for coupling with one or more medical treatment devices (e.g., a vacuum source (228) and a fluid source (230)). Valve body (902) is further configured to couple with a shaft assembly (908) to selectively provide fluid communication between end effector (not shown) of instrument (899). By way of example, valve assembly (900) may be configured similar to, and operable to, any of valve assemblies (226, 400, 500, 600, 700, 800). In some versions, valve assembly (900) may be secured within instrument housing (910) such that outlet shaft (912) of shaft assembly (908) may fluidly couple with another portion of shaft assembly (908) and/or an end effector (not shown) through an opening (814) through an exterior surface of instrument housing (810). In this respect, outlet shaft (912) is part of a disposable portion of surgical instrument (899) such that at least a portion of shaft assembly (908) is disposable with valve assembly (900), whereas a support sheath (913) of shaft assembly (908) extends from instrument housing (910) to support outlet shaft (912) in use. In some versions each of ports (904, 906) can include secure fittings such as, for example, luer fittings or any other similar connector operable to provide a leak-free fluid coupling. Outlet shaft (912) can couple with, for example, an end effector (not shown) of a surgical instrument to selectively provide fluids from the one or more medical treatment devices to end effector (not shown) as devised by valve assembly (900).

Depicted in FIG. 38 is disposable valve assembly (900) shown disassembled. Prior to performing a surgical operation with disposable portion (916) of surgical instrument (899) including disposable valve assembly (900) and disposable portion of shaft assembly (908), an operator may accordingly assemble disposable portion (916) and a reusable portion (918). Disposable portion (916) may thus include valve body (902), outlet shaft (912), and any internal valve components, and ports (904, 906). Reusable portion (918) may include all other remaining components of surgical instrument (899), such as instrument housing (910), end effector (not shown), and any drive components operable to manipulate valve assembly (900). After use, disposable portion (916) may be thrown away as opposed to being cleaned and sterilized for later use, while reusable portion may be cleaned and sterilized for later use.

### V. Exemplary Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

A surgical instrument, comprising: (a) a shaft assembly including a lumen; (b) a rotary drive member operatively connected to the shaft assembly and configured to rotate about a drive axis; and (c) a valve assembly operatively connected to the rotary drive member, the valve assembly including: (i) a valve body, including: (A) a first fluid inlet configured to receive a first fluid from a first fluid source, (B) a second fluid inlet configured to receive a second fluid from a second fluid source, and (C) an outlet in fluid communication with the lumen, and (ii) a valve plug received within the valve body and configured to rotate about a plug axis relative to the valve body to a first position, a second position, and a third position, wherein the valve plug has at least a first conduit configured to selectively fluidly communicate with each of the outlet and at least one of the first or second fluid inlets, wherein the valve body in the first position inhibit fluid communication from the first and second fluid inlets to the outlet, wherein the valve body in the second position fluidly connects the first fluid inlet to the outlet for fluid communication with the lumen, and wherein the valve body in the third position fluidly connects the second fluid inlet to the outlet for fluid communication with the lumen.

### Example 2

The surgical instrument of Example 1, wherein the drive axis and the plug axis are coaxial.

### Example 3

The surgical instrument of any one or more of Examples 1 through 2, wherein the shaft assembly extends along a longitudinal axis and further includes a linear actuator configured to selectively translate relative to the longitudinal axis, wherein the linear actuator is operatively connected to the rotary drive member and configured to selectively direct rotation of the rotary drive member.

### Example 4

The surgical instrument of Example 3, wherein the linear actuator includes at least one cable.

### Example 5

The surgical instrument of any one or more of Examples 1 through 4, wherein the valve plug is configured to rotate about the plug axis relative to the valve body to a fourth position, wherein the valve body in the fourth position inhibits fluid communication from the first and second fluid inlets to the outlet and fluidly connects the first fluid inlet to the second fluid inlet.

### Example 6

The surgical instrument of any one or more of Examples 1 through 5, wherein each of the first fluid inlet, the second fluid inlet, and the outlet extend perpendicular to the plug axis.

### Example 7

The surgical instrument of Example 6, wherein the plug axis extends in a first direction, and wherein the first fluid inlet is offset from at least one of the second fluid inlet or the outlet in the first direction.

### Example 8

The surgical instrument of Example 6, wherein each of the first fluid inlet, the second fluid inlet, and the outlet are positioned in a first plane.

### Example 9

The surgical instrument of any one or more of Examples 1 through 8, wherein each of the first fluid inlet, the second fluid inlet, and the outlet extend parallel to the plug axis.

### Example 10

The surgical instrument of any one or more of Examples 1 through 9, wherein the at least the first conduit further includes a second conduit.

### Example 11

The surgical instrument of Example 10, wherein the first and second conduit are fluidly disconnected from each other.

### Example 12

The surgical instrument of Example 10, wherein the at least the first conduit further includes a third conduit.

### Example 13

The surgical instrument of Example 12, wherein the first, second, and third conduit are fluidly connected as a common conduit.

### Example 14

The surgical instrument of Example 12, wherein the first, second, and third conduit and fluidly disconnected from each other.

### Example 15

The surgical instrument of any one or more of Examples 1 through 14, further comprising a reusable portion and a disposable portion, wherein the disposable portion is configured to removable connect to the reusable portion, and wherein the disposable portion includes the valve assembly and the reusable portion includes at least a portion of a body.

### Example 16

A robotic surgical system, comprising: (a) a patient support; (b) at least one robotic arm movable relative to the patient support; and (c) a surgical instrument comprising: (i) a shaft assembly including a lumen and a linear actuator, (ii) an end effector distally extending from the shaft assembly, wherein the end effector includes an opening fluidly connected to the lumen, (iii) a rotary drive member operatively connected to linear actuator and configured to be rotatably driven about a drive axis via the linear actuator; and (iv) a valve assembly operatively connected to the rotary drive member, the valve assembly including: (A) a valve body, including: (I) a first fluid inlet configured to receive a first fluid from a first fluid source, (II) a second fluid inlet configured to receive a second fluid from a second fluid source, and (III) an outlet in fluid communication with the lumen, and (B) a valve plug received within the valve body and configured to rotate about a plug axis relative to the valve body to a first position, a second position, and a third position, wherein the valve plug has at least a first conduit configured to selectively fluidly communicate with each of the outlet and at least one of the first or second fluid inlets, wherein the valve body in the first position inhibit fluid communication from the first and second fluid inlets to the outlet, wherein the valve body in the second position fluidly connects the first fluid inlet to the outlet for fluid communication with the lumen, and wherein the valve body in the third position fluidly connects the second fluid inlet to the outlet for fluid communication with the lumen.

### Example 17

The robotic surgical system of Example 16, wherein the drive axis and the plug axis are coaxial.

### Example 18

The robotic surgical system of any one or more of Examples 16 through 17, wherein the valve plug is configured to rotate about the plug axis relative to the valve body to a fourth position, wherein the valve body in the fourth position inhibits fluid communication from the first and second fluid inlets to the outlet and fluidly connects the first fluid inlet to the second fluid inlet.

### Example 19

A surgical instrument, comprising: (a) a reusable portion, including: (i) at least a portion of body, and (ii) an actuator; and (b) a disposable portion, including: (i) a valve assembly operatively connected to the actuator, the valve assembly including: (A) a valve body, including: (I) a first fluid inlet configured to receive a first fluid from a first fluid source, (II) a second fluid inlet configured to receive a second fluid from a second fluid source, and (III) an outlet configured to fluidly connect to the reusable portion.

### Example 20

The surgical instrument of Example 19, wherein the reusable portion further includes at least a portion of a shaft assembly.

### VI. Miscellaneous

Any one or more of the teaching, expressions, embodiments, examples, etc. described herein may be combined with any one or more of the teachings, expressions, embodiments, examples, etc. described in U.S. Pat. App. No. [Atty. Ref. No. END9330USNP1], entitled “Suction and Irrigation Valve and Method of Priming Same in a Robotic Surgical System,” filed on even date herewith and incorporated by reference in its entirety herein.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Versions described above may be designed to be disposed of after a single use, or they can be designed to be used multiple times. Versions may, in either or both cases, be reconditioned for reuse after at least one use. Reconditioning may include any combination of the steps of disassembly of the systems, instruments, and/or portions thereof, followed by cleaning or replacement of particular pieces, and subsequent reassembly. In particular, some versions of the systems, instruments, and/or portions thereof may be disassembled, and any number of the particular pieces or parts of the systems, instruments, and/or portions thereof may be selectively replaced or removed in any combination. Upon cleaning and/or replacement of particular parts, some versions of the systems, instruments, and/or portions thereof may be reassembled for subsequent use either at a reconditioning facility, or by an operator immediately prior to a procedure. Those skilled in the art will appreciate that reconditioning of systems, instruments, and/or portions thereof may utilize a variety of techniques for disassembly, cleaning/replacement, and reassembly. Use of such techniques, and the resulting reconditioned systems, instruments, and/or portions thereof, are all within the scope of the present application.

By way of example only, versions described herein may be sterilized before and/or after a procedure. In one sterilization technique, the systems, instruments, and/or portions thereof is placed in a closed and sealed container, such as a plastic or TYVEK bag. The container and system, instrument, and/or portion thereof may then be placed in a field of radiation that can penetrate the container, such as gamma radiation, x-rays, or high-energy electrons. The radiation may kill bacteria on the system, instrument, and/or portion thereof and in the container. The sterilized systems, instruments, and/or portions thereof may then be stored in the sterile container for later use. Systems, instruments, and/or portions thereof may also be sterilized using any other technique known in the art, including but not limited to beta or gamma radiation, ethylene oxide, or steam.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. A surgical instrument, comprising:
(a) a shaft assembly including a lumen;
(b) a rotary drive member operatively connected to the shaft assembly and configured to rotate about a drive axis; and
(c) a valve assembly operatively connected to the rotary drive member, the valve assembly including:
(i) a valve body, including:
(A) a first fluid inlet configured to receive a first fluid from a first fluid source,
(B) a second fluid inlet configured to receive a second fluid from a second fluid source, and
(C) an outlet in fluid communication with the lumen, and
(ii) a valve plug received within the valve body and configured to rotate about a plug axis relative to the valve body to a first position, a second position, and a third position, wherein the valve plug has at least a first conduit configured to selectively fluidly communicate with each of the outlet and at least one of the first or second fluid inlets, wherein the valve body in the first position inhibit fluid communication from the first and second fluid inlets to the outlet, wherein the valve body in the second position fluidly connects the first fluid inlet to the outlet for fluid communication with the lumen, and wherein the valve body in the third position fluidly connects the second fluid inlet to the outlet for fluid communication with the lumen.

2. The surgical instrument of claim 1, wherein the drive axis and the plug axis are coaxial.

3. The surgical instrument of claim 1, wherein the shaft assembly extends along a longitudinal axis and further includes a linear actuator configured to selectively translate relative to the longitudinal axis, wherein the linear actuator is operatively connected to the rotary drive member and configured to selectively direct rotation of the rotary drive member.

4. The surgical instrument of claim 3, wherein the linear actuator includes at least one cable.

5. The surgical instrument of claim 1, wherein the valve plug is configured to rotate about the plug axis relative to the valve body to a fourth position, wherein the valve body in the fourth position inhibits fluid communication from the first and second fluid inlets to the outlet and fluidly connects the first fluid inlet to the second fluid inlet.

6. The surgical instrument of claim 1, wherein each of the first fluid inlet, the second fluid inlet, and the outlet extend perpendicular to the plug axis.

7. The surgical instrument of claim 6, wherein the plug axis extends in a first direction, and wherein the first fluid inlet is offset from at least one of the second fluid inlet or the outlet in the first direction.

8. The surgical instrument of claim 6, wherein each of the first fluid inlet, the second fluid inlet, and the outlet are positioned in a first plane.

9. The surgical instrument of claim 1, wherein each of the first fluid inlet, the second fluid inlet, and the outlet extend parallel to the plug axis.

10. The surgical instrument of claim 1, wherein the at least the first conduit further includes a second conduit.

11. The surgical instrument of claim 10, wherein the first and second conduit are fluidly disconnected from each other.

12. The surgical instrument of claim 10, wherein the at least the first conduit further includes a third conduit.

13. The surgical instrument of claim 12, wherein the first, second, and third conduit are fluidly connected as a common conduit.

14. The surgical instrument of claim 12, wherein the first, second, and third conduit and fluidly disconnected from each other.

15. The surgical instrument of claim 1, further comprising a reusable portion and a disposable portion, wherein the disposable portion is configured to removable connect to the reusable portion, and wherein the disposable portion includes the valve assembly and the reusable portion includes at least a portion of a body.

16. A robotic surgical system, comprising:
(a) a patient support;
(b) at least one robotic arm movable relative to the patient support; and
(c) a surgical instrument comprising:
(i) a shaft assembly including a lumen and a linear actuator,
(ii) an end effector distally extending from the shaft assembly, wherein the end effector includes an opening fluidly connected to the lumen,
(iii) a rotary drive member operatively connected to linear actuator and configured to be rotatably driven about a drive axis via the linear actuator; and
(iv) a valve assembly operatively connected to the rotary drive member, the valve assembly including:
(A) a valve body, including:
(I) a first fluid inlet configured to receive a first fluid
(II) a second fluid inlet configured to receive a second fluid from a second fluid source, and
(III) an outlet in fluid communication with the lumen, and
(B) a valve plug received within the valve body and configured to rotate about a plug axis relative to the valve body to a first position, a second position, and a third position, wherein the valve plug has at least a first conduit configured to selectively fluidly communicate with each of the outlet and at least one of the first or second fluid inlets, wherein the valve body in the first position inhibit fluid communication from the first and second fluid inlets to the outlet, wherein the valve body in the second position fluidly connects the first fluid inlet to the outlet for fluid communication with the lumen, and wherein the valve body in the third position fluidly connects the second fluid inlet to the outlet for fluid communication with the lumen.

17. The robotic surgical system of claim 16, wherein the drive axis and the plug axis are coaxial.

18. The robotic surgical system of claim 16, wherein the valve plug is configured to rotate about the plug axis relative to the valve body to a fourth position, wherein the valve body in the fourth position inhibits fluid communication from the first and second fluid inlets to the outlet and fluidly connects the first fluid inlet to the second fluid inlet.

19. A surgical instrument, comprising:
(a) a reusable portion, including:
(i) at least a portion of body, and
(ii) an actuator; and
(b) a disposable portion, including:
(i) a valve assembly operatively connected to the actuator, the valve assembly including:
(A) a valve body, including:
(I) a first fluid inlet configured to receive a first fluid from a first fluid source,
(II) a second fluid inlet configured to receive a second fluid from a second fluid source, and
(III) an outlet configured to fluidly connect to the reusable portion.

20. The surgical instrument of claim 19, wherein the reusable portion further includes at least a portion of a shaft assembly.
